(19) United States
(12) Patent Application Publication
(10) Pub. No.: US 2026/0242440 A1
(43) Pub. Date: Aug. 20, 2026

(54) NOVEL MEANS TO MODULATE NMDA RECEPTOR-MEDIATED TOXICITY

(71) Applicant: FUNDAMENTAL PHARMA GMBH, Heidelberg (DE)

(72) Inventors: Hilmar BADING, Heidelberg (DE); Jing YAN, Heidelberg (DE)

(73) Assignee: FUNDAMENTAL PHARMA GMBH, Heidelberg (DE)

(21) Appl. No.: 19/552,174

(22) Filed: Feb. 27, 2026

Related U.S. Application Data
(63) Continuation of application No. 17/286,207, filed on Oct. 18, 2019, now abandoned.

(30) Foreign Application Priority Data
Oct. 18, 2018 (WO) PCT/EP2018/078577
Apr. 29, 2019 (WO) PCT/EP2019/060890

(51) Int. Cl. C07K 14/705; A61K 9/51; A61K 31/137; A61K 31/40; A61K 31/454; A61K 38/00; A61K 45/06; G16B 15/30

(52) U.S. Cl. CPC C07K 14/705; A61K 31/137; A61K 31/40; A61K 31/454; A61K 45/06; G16B 15/30; A61K 9/51; A61K 38/00; C07K 2319/00

(57) ABSTRACT

The present invention relates to the field of neurodegenerative processes and means to provide protection against the same. In particular, the present invention relates to polypeptides, fusion proteins, and other compounds interacting with the N-terminal domain of transient receptor potential melastatin subfamily member 4 (TRPM4), which are capable of interfering with NMDA receptor mediated neurotoxicity. The present invention also relates to nucleic acids encoding the aforementioned polypeptides or fusion proteins, compositions comprising the same and the use of said polypeptides, fusion proteins, and other compounds in methods for treating or preventing a disease of the human or animal body, for example in a method of treating diseases like Alzheimer's disease (AD), amyotrophic lateral sclerosis (ALS), Huntington's disease (HD) or stroke.

### CROSS-REFERENCE TO RELATED APPLICATIONS

This application is continuation of U.S. patent application Ser. No. 17/286,207, filed on Apr. 16, 2021, which is a national phase application under 35 U.S.C. § 371 of International Application No. PCT/EP2019/078415, filed on Oct. 18, 2019, which claims benefit of priority to International Application Nos. PCT/EP2018/078577, filed on Oct. 18, 2018, and PCT/EP2019/060890, filed on Apr. 29, 2019. The entire contents of each of the aforementioned application is hereby incorporated by reference.

### INCORPORATION OF SEQUENCE LISTING

This application contains a Sequence Listing XML, which has been submitted electronically and is hereby incorporated by reference in its entirety. Said Sequence Listing XML, created on Apr. 23, 2026, is named DEBEP0155USC1.xml and is 70,534 bytes in size.

The present invention relates to the field of neurodegenerative processes and means to provide protection against the same. In particular, the present invention relates to polypeptides, fusion proteins, and other compounds interacting with the N-terminal domain of transient receptor potential melastatin subfamily member 4 (TRPM4), which are capable of interfering with NMDA receptor mediated neurotoxicity. The present invention also relates to nucleic acids encoding the aforementioned polypeptides or fusion proteins, compositions comprising the same and the use of said polypeptides, fusion proteins, and other compounds in methods for treating or preventing a disease of the human or animal body, for example in a method of treating diseases like Alzheimer's disease (AD), amyotrophic lateral sclerosis (ALS), Huntington's disease (HD) or stroke.

Neurodegenerative diseases are devastating diseases involving the progressive loss of structure or function of neurons and eventual death of neurons. Neurodegeneration may be acute or slowly progressive, but both types of neurodegeneration often involve increased death signaling by extrasynaptic NMDA receptors caused by elevated extracellular glutamate concentrations or relocalization of NMDA receptors to extrasynaptic sites. NMDA receptors are glutamate- and voltage-gated ion channels that are permeable for calcium. They can be categorized according to their subcellular location as synaptic and extrasynaptic NMDA receptors. The subunit composition of the receptors within and outside synaptic contacts is similar, although, in addition to carrying the common Glutamate Ionotropic Receptor NMDA Type Subunit 1 (GRIN1) subunit, extrasynaptic NMDA receptors contain preferentially the GRIN2B subunit, whereas GRIN2 Å is the predominant subunit in synaptic NMDA receptors.

The cellular consequences of synaptic versus extrasynaptic NMDA receptor stimulation are dramatically different. Synaptic NMDA receptors initiate physiological changes in the efficacy of synaptic transmission. They also trigger calcium signaling pathways to the cell nucleus that activate gene expression responses critical for the long-term implementation of virtually all behavioral adaptations. Most importantly, synaptic NMDA receptors, acting via nuclear calcium, are strong activators of neuronal structure-protective and survival-promoting genes. In striking contrast, extrasynaptic NMDA receptors trigger cell death pathways. Within minutes after extrasynaptic NMDA receptor activation, the mitochondrial membrane potential breaks down, followed by mitochondrial permeability transition. Extrasynaptic NMDA receptors also strongly antagonize excitation-transcription coupling and disrupt nuclear calcium-driven adaptogenomics because they trigger a cyclic adenosine monophosphate (cAMP)-responsive element-binding protein (CREB) shutoff pathway, inactivate extracellular signal-regulated kinase (ERK)-MAPK signaling, and lead to nuclear import of class IIa histone deacetylases (HDACs) and the pro-apoptotic transcription factor Foxo3A. This affects activity regulation of many genes, including brain-derived neurotrophic factor (bdnf) and vascular endothelial growth factor D (vegfd), that are vital for the maintenance of complex dendritic architecture and synaptic connectivity as well as the buildup of a neuroprotective shield. In addition, given the short reach of activated ERK1/2, their shut-off by extrasynaptic NMDA receptors disrupts important local signaling events including dendritic mRNA translation and AMPA (α-amino-3-hydroxy-5-methyl-4-isoxazolepropionic acid) receptor trafficking that controls the efficacy of synaptic transmission.

Thus, extrasynaptic NMDA receptor signaling is characterized by the initiation of a pathological triad with mitochondrial dysfunction, deregulation of transcription, and loss of integrity of neuronal structures and connectivity.

Several attempts have been made to use blockers of NMDA receptors for treatments of neurological conditions. In general, the results of clinical studies were disappointing largely because of serious side effects caused by interference of the blockers with the physiological function of synaptically localized NMDA receptors (Ogden and Traynelis, 2011). One notable exception is the NMDA receptor antagonist memantine (Bormann, 1989). Beneficial effects of low-dose treatments with memantine have been observed in several animal models of neurodegeneration, which include Alzheimer's disease (AD), Huntington's disease (HD), amyotrophic lateral sclerosis (ALS), and the experimental autoimmune encephalomyelitis (EAE) model of MS. Moreover, memantine is approved since 2002 by the European Medicines Agency and the US Food and Drug Administration (FDA) for the treatment of AD. The discovery that memantine in a certain concentration range blocks preferentially the toxic extrasynaptic NMDA receptors explains why it is effective in a wide range of neurodegenerative conditions that share toxic extrasynaptic NMDA receptor signaling as a pathomechanism (Bading, J Exp Med. 2017 Mar. 6; 214(3):569-578).

Thus, means of selectively attenuating specifically the toxic activity of extrasynaptic NMDA receptors hold great potential for the development of broadly effective, well-tolerated neuroprotective therapeutics and there is still a need in the art for such new means. The problem to be solved by the present invention was thus to provide new means to attenuate extrasynaptic toxic NMDA receptor activity, thereby allowing for improved (since being preferably more selective) treatment of neurodegenerative diseases like Alzheimer's disease (AD), amyotrophic lateral sclerosis (ALS), Huntington's disease (HD), or stroke.

This problem is solved by the subject-matter as set forth in the appended claims and in the description below.

The inventors of the present invention have surprisingly found that NMDA receptor mediated toxicity can be selectively inhibited without significant impact on synaptic NMDA signaling. The compounds for use of the invention mimic a portion of the N-terminal domain of transient receptor potential melastatin subfamily member 4 (TRPM4) or bind to and/or form a complex with the N-terminal domain of TRPM4 (and, without being bound by this theory, block thereby interaction of the extrasynaptic NMDA receptor complex with the N-terminal domain of TRPM4). Two different isoforms of human TRPM4 are depicted in SEQ ID NO:1 and SEQ ID NO:2. The selective protection conferred against NMDA receptor mediated cytotoxicity allows for treatment and prevention of neuronal and in particular of neurodegenerative diseases. Therefore, the present invention relates in a first aspect to a polypeptide comprising a fragment of human TRPM4, namely comprising an amino acid sequence according to SEQ ID NO:3, or comprising a derivative of said sequence according to SEQ ID NO:3. Human TRPM4 comprises a cytosolic N-terminal domain, a transmembrane domain, and a cytosolic C-terminal domain.

SEQ ID NO:3 is the C-terminal portion of the N-terminal domain of human TRPM4, corresponding to amino acids 633-689 of the human TRPM4 sequence (see the isoforms of SEQ ID NO:1 and SEQ ID NO:2, which are fully conserved in the N-terminal domain). The polypeptide of the invention may comprise aside of SEQ ID NO:3, or its derivative, further TRPM4 derived sequences, in particular sequences flanking SEQ ID NO:3 in (e.g., human) TRPM4. For example, the polypeptide may comprise additional N-terminal sequences, such as amino acids 347-632 of (e.g., human) TRPM4. However, since the polypeptide of the present invention is defined as comprising a fragment of TRPM4, a polypeptide of the invention will not comprise the sequence of a full length (e.g. human) TRPM4 protein. As used herein, “TRPM4 protein” refers to a full length sequence of transient receptor potential melastatin subfamily member 4 known to the person skilled in the art. For example, the two isoforms SEQ ID NO:1 and SEQ ID NO:2 are human TRPM4 proteins. The term encompasses also all orthologues of human TRPM4 proteins known from other species, such as mouse. Examples of species with known TRPM4 sequences are listed in table 1, left column. A polypeptide according to the present invention does not comprise the full length amino acid sequence of human transient receptor potential melastatin subfamily member 4 (TRPM4), irrespective of the isoform, nor will it comprise the full length amino acid sequence of TRPM4 orthologues of other species.

Preferably, the polypeptide of the invention will also not comprise the sequence of a functional fragment of a TRPM4 protein (irrespective of isoform or species of origin). A “functional fragment of TRPM4” is a fragment of a TRPM4 protein that retains the biologic activity of TRPM4, i.e., is still capable of forming a and acting as cation channel, thereby regulating the influx of cations such as Na+. Techniques to measure channel activity are generally known in the art and channel activity can be easily measured, for example in HEK293 cells transfected with expression vectors for TRPM4 or the respective fragment(s) of TRPM4. An appropriate technique is for example disclosed in Amarouch et al., Neurosci Lett. 2013 Apr. 29; 541:105-10.

More preferably, the polypeptide does not comprise one or both of the C-terminal domain of a human TRPM4 protein and the transmembrane domain of a human TRPM4 protein. More preferably, the polypeptide does neither comprise the C-terminal domain of a human TRPM4 protein nor the transmembrane domain of human TRPM4 protein (irrespective of isoform). Most preferably, any human TRPM4 derived sequence within the inventive polypeptide is limited to a fragment of the N-terminal domain of a TRPM4 protein, in particular to amino acids 633-689 of the human TRPM4 sequence.

The polypeptide may also comprise instead of SEQ ID NO:3 a derivative of SEQ ID NO:3. An example for a derivative of the sequence according to SEQ ID NO:3 is a sequence falling within consensus sequence according to SEQ ID NO:4, with the proviso that the sequence is not SEQ ID NO:3. SEQ ID NO:4 is a consensus sequence of the C-terminal portion of the N-terminal domain of TRPM4 proteins of various mammalian species, as shown in table 1 below:

TABLE 11
Inventive TRPM4 motif in 38 mammalian species

| | | | SEQ |
|---|---|---|---|
| | | | ID |
| | Species | Sequence | NO: |
| 1 | Mus musculus | NSEERAARLLLRRCPLWGEATCLQLAMQADARAFFAQDGVQSLLTQK | 5 |
| | | WWGEMDSTTP | |
| 2 | Peromyscus | NSEERAARLLLRRCPLWGEATCLQLAMQADARAFFAQDGVQSLLTQK | 6 |
| | maniculatus | WWGEMDSTTP | |
| 3 | Mus caroli | NSEDRAARLLLRRCPLWGEATCLQLAMQADARAFFAQDGVQSLLTQK | 7 |
| | | WWGEMDSTTP | |
| 4 | Cricetulus | NSEERAARLLLRRCPLWGEATCLQLAMQADARSFFAQDGVQSLLTQK | 8 |
| | griseus | WWGDMDSTTP | |
| 5 | Jaculus jaculus | NNEDRAARLLLRRCPLWGEATCLQLAMQADARAFFAQDGVQSLLTQK | 9 |
| | | WWGEMDSTTP | |
| 6 | Canis lupus | SSEERAARLLLRRCPLWGDATCLQLAMQADARAFFAQDGVQSLLTQK | 10 |
| | familiaris | WWGEMDSTTP | |
| 7 | Hipposideros | NSEDRAARLLLRRCPFWGDATCLQLAMQADARAFFAQDGVQSLLTQK | 11 |
| | armiger | WWGEMDSTTP | |
| 8 | Rattus | NSEYRAARLLLRRCPLWGEATCLQLAMQADARAFFAQDGVQSLLTQK | 12 |
| | norvegicus | WWGEMDSTNP | |
| 9 | Acinonyx | SSEKRAARLLLRRCPLWGDATCLQLAMQADARAFFAQDGVQSLLTQK | 13 |
| | jubatus | WWGEMDSTTP | |
| 10 | Odobenus | SSEERAARLLVRRCPLWGDATCLQLAMQADARAFFAQDGVQSLLTQK | 14 |
| | rosmarus | WWGEMDSTTP | |
| | divergens | | |
| 11 | Neomonachus | SSEERAARLLVRRCPLWGDATCLQLAMQADARAFFAQDGVQSLLTQK | 15 |
| | schauinslandi | WWGEMDSTTP | |
| 12 | Manis javanica | SSEHRAARLLIRRCPLWGDATCLQLAMQADARAFFAQDGVQSLLTQK | 16 |
| | | WWGEMDSTTP | |
| 13 | Marmota | SSEDRAARLLLRRCPLWGDATCLQLAMQADARAFFAQDGVQSLLTQK | 17 |
| | marmota | WWGEMDSTTP | |
| | marmota | | |
| 14 | Enhydra lutris | SSEKRAARLLLRRCPLWGDATCLQLAMQADARAFFAQDGVQSLLTQK | 18 |
| | kenyoni | WWGEMDSTTP | |
| 15 | Ictidomys | SSEDRAARLLLRRCPLWGDATCLQLAMQADARAFFAQDGVQSLLTQK | 19 |
| | tridecemlineatus | WWGEMDSTTP | |
| 16 | Felis catus | SSEKRAARLLLRRCPLWGDATCLQLAMQADARAFFAQDGVQSLLTQK | 20 |
| | | WWGEMDSTTP | |
| 17 | Heterocephalus | SSEERASRLLLRRCPLWGDATCLQLAMQADARAFFAQDGVQSLLTQK | 21 |
| | glaber | WWGEMDSTTP | |
| 18 | Capra hircus | SSEERSARLLLRRCPLWGDATCLQLATQADARAFFAQDGVQSLLTQKW | 22 |
| | | WGEMDSTTP | |
| 19 | Equus asinus | SSEERASRLLLRRCPLWGDATCFQLAMQADARAFFAQDGIQSLLTQKW | 23 |
| | | WGEMDSTTP | |
| 20 | Rhinolophus | NSEDRAARLLLRRCPFWGDATCFQLAMQADARAFFAQDGVQSLLTQK | 24 |
| | sinicus | WWGEMDSSTP | |
| 21 | Ovis aries | SSEERSARLLLRRCPLWGDATCLQLATQADARAFFAQDGVQSLLTQKW | 25 |
| | | WGEMDSTTP | |
| 22 | Mesocricetus | NSEERAAGLLLRRCPLWGEATCLQLAMQADARSFFAQDGVQFLLTQK | 26 |
| | auratus | WWGEMDSTTP | |
| 23 | Bison bison | SSEERSARLLLRRCPLWGDATCLQLATQADARAFFAQDGVQSLLTQKW | 27 |
| | bison | WGEMDSTTP | |
| 24 | Bos taurus | SSEERSARLLLRRCPLWGDATCLQLATQADARAFFAQDGVQSLLTQKW | 28 |
| | | WGEMDSTTP | |
| 25 | Bubalus bubalis | SSEERSARLLLRRCPLWGDATCLQLATQADARAFFAQDGVQSLLTQKW | 29 |
| | | WGEMDSTTP | |
| 26 | Erinaceus | SSEDRANRLLLRRCPLWGDATCLQLAMQADARAFFAQDGVQSLLTQK | 30 |
| | europaeus | WWGEMDSTTP | |
| 27 | Cavia porcellus | SNEHRASRLLLRRCPLWGDATCLQLAMQADSRAFFAQDGVQSLLTQK | 31 |
| | | WWGEMDSTTP | |
| 28 | Equus caballus | SSEERASRLLLRRCPLWGGATCFQLAMQADARAFFAQDGIQSLLTQKW | 32 |
| | | WGEMDSTTP | |
| 29 | Camelus | SSEDRAARLLLRRCPLWGDSTCLQLATQADARAFFAQDGVQSLLTQK | 33 |
| | dromedarius | WWGEMDSTTP | |
| 30 | Homo sapiens | SSEVRAARLLLRRCPLWGDATCLQLAMQADARAFFAQDGVQSLLTQK | 3 |
| | | WWGDMASTTP | |
| 31 | Theropithecus | SSEVRAARLLLRRCPLWGDATCLQLAMQADARAFFAQDGVQSLLTQK | 34 |
| | gelada | WWGDMASTTP | |
| 32 | Orcinus orca | SSEERAAHLLLWRCPLWGDATCLHLAMQADARAFFAQDGVQSLLTQK | 35 |
| | | WWGEMDSTTP | |
| 33 | Chlorocebus | SSEVRAARLLLRRCPLWGDATCLQLAMQADARAFFAQDGVQSLLTQK | 36 |
| | sabaeus | WWGDMASTTP | |
| 34 | Chinchilla | SSETRASRLLLRRCPLWGDATCLQLAMQADARAFLAQDGVQSVLTQK | 37 |
| | lanigera | WWGEMDSTTP | |
| 35 | Elephantulus | SNEKWAARLLLRRCPLWGDATCLQLAMQADSRAFFAQDGVQSLLTQK | 38 |
| | edwardii | WWGEMDSTTP | |
| 36 | Gorilla gorilla | SNEVRAARLLLRRCPLWGDATCLQLAMQADARAFFAQDGVQSLLTQK | 39 |
| | gorilla | WWGDMASTTP | |
| 37 | Sus scrofa | NSENRAARLLLRRCPLWGDATCLQLATQADARAFFAQDGVQSLLTQK | 40 |
| | | WWGHMDSTTP | |
| 38 | Pan troglodytes | SSEVRAARLLLRRCPLWGDATCLQLAMQADARAFFAQDGVQSLLTQK | 41 |
| | | WWGDMASTTP | |

As evident from table 1 above, the motif of interest is well conserved across various mammalian species. The human motif, SEQ ID NO:3, is 100% conserved in between Homo sapiens, Theropithecus gelada, Chlorocebus sabaeus and Pan troglodytes. And none of the other mammalian species deviates by more than 20% from the human sequence. A polypeptide comprising a derivative of human SEQ ID NO:3, wherein the derivative is derived from another mammalian species, will typically be used for methods of treatment of a subject of the corresponding species (see also ninth aspect and tenth aspect of the present invention further down below). However, the inventors have shown that for example a sequence derived from mouse TRPM4 can be used with similar effects in human cell line HEK293 as in mice, indicating the conserved function, and thus utility, of the inventive polypeptide across mammalian species borders. In a preferred embodiment of the invention, the derivative of SEQ ID NO:3 is thus SEQ ID NO:5.

The derivative of human SEQ ID NO:3 may also be a sequence selected from the group consisting of a sequence having at least 80% sequence identity with SEQ ID NO:5, a sequence having at least 80% sequence identity with SEQ ID NO:6, a sequence having at least 80% sequence identity with SEQ ID NO:7, a sequence having at least 80% sequence identity with SEQ ID NO:8, a sequence having at least 80% sequence identity with SEQ ID NO:9, a sequence having at least 80% sequence identity with SEQ ID NO:10, a sequence having at least 80% sequence identity with SEQ ID NO:11, a sequence having at least 80% sequence identity with SEQ ID NO:12, a sequence having at least 80% sequence identity with SEQ ID NO:13, a sequence having at least 80% sequence identity with SEQ ID NO:14, a sequence having at least 80% sequence identity with SEQ ID NO:15, a sequence having at least 80% sequence identity with SEQ ID NO:16, a sequence having at least 80% sequence identity with SEQ ID NO:17, a sequence having at least 80% sequence identity with SEQ ID NO:18, a sequence having at least 80% sequence identity with SEQ ID NO:19, a sequence having at least 80% sequence identity with SEQ ID NO:20, a sequence having at least 80% sequence identity with SEQ ID NO:21, a sequence having at least 80% sequence identity with SEQ ID NO:22, a sequence having at least 80% sequence identity with SEQ ID NO:23, a sequence having at least 80% sequence identity with SEQ ID NO:24, a sequence having at least 80% sequence identity with SEQ ID NO:25, a sequence having at least 80% sequence identity with SEQ ID NO:26, a sequence having at least 80% sequence identity with SEQ ID NO:27, a sequence having at least 80% sequence identity with SEQ ID NO:28, a sequence having at least 80% sequence identity with SEQ ID NO:29, a sequence having at least 80% sequence identity with SEQ ID NO:30, a sequence having at least 80% sequence identity with SEQ ID NO:31, a sequence having at least 80% sequence identity with SEQ ID NO:32, a sequence having at least 80% sequence identity with SEQ ID NO:33, a sequence having at least 80% sequence identity with SEQ ID NO:34, a sequence having at least 80% sequence identity with SEQ ID NO:35, a sequence having at least 80% sequence identity with SEQ ID NO:36, a sequence having at least 80% sequence identity with SEQ ID NO:37, a sequence having at least 80% sequence identity with SEQ ID NO:38, a sequence having at least 80% sequence identity with SEQ ID NO:39, a sequence having at least 80% sequence identity with SEQ ID NO:40, and a sequence having at least 80% sequence identity with SEQ ID NO:41.

As used herein, the term “% sequence identity”, has to be understood as follows: Two sequences to be compared are aligned to give a maximum correlation between the sequences.

This may include inserting “gaps” in either one or both sequences, to enhance the degree of alignment. A % identity may then be determined over the whole length of the aligned sequences being compared, including potential gaps. In the above context, an amino acid sequence having a “sequence identity” of at least, for example, 95% to a reference amino acid sequence, is intended to mean that the sequence of the reference amino acid sequence is identical to the query sequence except that the query amino acid sequence may include up to five amino acid residue alterations (substitutions, deletions, insertions) per each 100 amino acids of the reference amino acid sequence. Methods for comparing the identity of two or more sequences are well known in the art. The percentage to which two sequences are identical can for example be determined by using a mathematical algorithm. A preferred, but not limiting, example of a mathematical algorithm which can be used is the algorithm of Karlin et a/. (1993), PNAS USA, 90:5873-5877. Such an algorithm is integrated in the BLAST family of programs, e.g. BLAST or NBLAST program (see also Altschul et al., 1990, J. Mol. Biol. 215, 403-410 or Altschul et al. (1 997), Nucleic Acids Res, 25:3389-3402), accessible through the home page of the NCBI at world wide web site ncbi.nlm.nih.gov) and FASTA (Pearson (1 990), Methods Enzymol. 83, 63-98; Pearson and Lipman (1988), Proc. Natl. Acad. Sci. U.S.A 85, 2444-2448.). Sequences which are identical to other sequences to a certain extent can be identified by these programmes. Furthermore, programs available in the Wisconsin Sequence Analysis Package, version 9.1 (Devereux et al, 1984, Nucleic Acids Res., 387-395), for example the programs BESTFIT and GAP, may be used to determine the % identity between two polypeptide sequences. If herein reference is made to an amino acid sequence sharing a particular extent of sequence identity to a reference sequence, then said difference in sequence is preferably due to conservative amino acid substitutions. Preferably, such sequence retains the function and activity of the reference sequence, albeit maybe to a higher or lower degree. In addition, if reference is made herein to a sequence sharing “at least” a certain percentage of sequence identity, then 100% sequence identity are preferably not encompassed.

Wherever herein reference is made to a sequence having at least 80% sequence identity with a respective SEQ ID NO:, e.g. SEQ ID NO:3, said sequence may have for example at least 81%, at least 83%, at least 85%, at least 86%, at least 88%, at least 90%, at least 92%, at least 93%, at least 95%, or at least 97% sequence identity with the respective reference SEQ ID NO:, e.g. SEQ ID NO:3. In those cases where the reference sequence is not SEQ ID NO:3, the derivative may also have 100% sequence identity with the respective reference sequence. For example, the derivative of SEQ ID NO:3 may be a sequence having 100% sequence identity with SEQ ID NO:5, i.e. may be the respective TRPM4 sequence of the mouse. A derivative of SEQ ID NO:3, in particular any sequence comprising at least 80% sequence identity with of SEQ ID NO:3 or comprising at least 80% sequence identity with any of the respective sequences of other species listed in table 1, may contain mutations, preferably conservative mutations (i.e. mutations reflecting an amino acid replacement that changes a given amino acid residue of SEQ ID NO:3 to a different amino acid with similar biochemical properties (e.g. charge, hydrophobicity and size)). For example, any one of the two phenylalanine residues at positions 34 and 35 of SEQ ID NO:3, or both, may be substituted by tyrosine (i.e. a replacement of an aromatic amino acid for another aromatic amino acid) without abrogation of the neuroprotective effect of the inventive polypeptide. The inventors consider similar mutations possible in the corresponding sequences of the other species, as said twin phenylalanine motif is conserved throughout all mammalian species listed in table 1, except for the chinchilla, which has a leucine at position 35. Leucine is thus also likely to be an acceptable amino acid substitution at position 35 of SEQ ID NO:3. In the alternative, or in addition, a derivative may lack one or more amino acids at the N- and/or C-terminus of SEQ ID NO:3. For example, the derivative may lack 1, 2, 3, 4, 5, 6, 7, 8, 9, 10, 11 or 12 amino acids at the N- and/or C-terminus of SEQ ID NO:3 or its derivative, preferably lack 1, 2, 3, 4, 5, amino acids at the N- and/or C-terminus of SEQ ID NO:3 or its derivative.

It is understood that embodiments of the inventive polypeptide in which the derivative of the sequence according to SEQ ID NO:3 is a sequence falling within the consensus sequence according to SEQ ID NO:4, or is a sequence sharing at least 80% sequence identity with any of the respective sequences of species listed in table 1, that the claimed polypeptide will not comprise the full length amino acid sequence of orthologues to human TRPM4, just as it will not comprise the full length human TRPM4 sequence (see above). Similarly, what has been set out above regarding the presence and absence of other elements of TRPM4, such as flanking sequences or the C-terminal or transmembrane domain, applies likewise in analogous manner to polypeptides in which the derivative of the sequence according to SEQ ID NO:3 is a sequence falling within consensus sequence according to SEQ ID NO:4 or is a sequence sharing at least 80% sequence identity with any of the respective sequences of species listed in table 1, i.e. such elements may be present but are preferably not present.

Preferably, the polypeptide of the present invention is neuroprotective. As used herein, a compound is “neuroprotective”, if said compound protects both in vitro and in vivo against cell death evoked by harmful conditions. A standard in vitro test involves treatment of primary hippocampal or cortical neurons with NMDA for 10 minutes followed by assessments of cell death 24 hours later (see for example FIG. 3 c in Zhang et al., 2011, Neurosci. 31, 4978-4990). A standard in vivo test is the middle cerebral artery occlusion (MCAO) mouse stroke model (see for example FIG. 6 in Zhang et al., 2011). Statistically relevant differences in the rate of cell death measured in vitro or brain damage in vivo (given as infarct volume) compared to appropriate controls (i.e., saline solution, solvent only, inactive mutants) indicate neuroprotection.

Preferably, the length of a polypeptide according to the present invention will not exceed 685 amino acids in length. The inventive polypeptide may for instance be at most about 650 amino acids long, at most about 600 amino acids long, at most about 500 amino acids long, at most about 400 amino acids long, at most about 350 amino acids long, at most about 325 amino acids long, at most about 300 amino acids long, at most about 250 amino acids long, at most about 200 amino acids long, at most about 175 amino acids long, at most about 150 amino acids long, at most about 125 amino acids long, at most about 100 amino acids long, at most about 90 amino acids long, at most about 85 amino acids long, at most about 80 amino acids long, at most about 75 amino acids long, at most about 70 amino acids long, at most about 65 amino acids long, at most about 60 amino acids long.

In a second aspect, the present invention relates to a polypeptide binding to a polypeptide of the first aspect of the invention and/or binding to a full length TRPM4 in the corresponding region (i.e. SEQ ID NO:3 or its derivative). A polypeptide according to the second aspect of the present invention is also preferably neuroprotective. Preferably, the polypeptide is an antibody or anticalin. Even more preferably the polypeptide of this aspect is an antibody. Preferably, such antibody is not a rabbit anti-TRPM4 antibody.

In a third aspect, the present invention relates to a fusion protein comprising the inventive polypeptide according to the first aspect of the invention and at least one further (functional) amino acid sequence element heterologous to the amino acid sequence according to SEQ ID NO:3 or its derivative. “Heterologous” in this context means preferably, that the at least one further sequence does not occur in nature as a fusion with the amino acid sequence according to SEQ ID NO:3 or its derivative amino acid sequence. As a consequence, the resulting fusion protein is a non-naturally occurring, artificially created polypeptide. To be more precise, the amino acid sequence resulting from this fusion does not occur in this form in nature. The at least one heterologous amino acid sequence element may be at least 5, at least 10, at least 15 at least 20, at least 25, at least 30 at least 50, at least 100 amino acids, at least 250 amino acids, or at least 500 or more amino acids in length. For example, the further amino acid sequence may be selected from the group consisting of a membrane anchoring moiety, a protein transduction domain and a tag. Particularly preferred membrane anchoring moieties are selected from the group consisting of a CaaX box motif (for prenylation), a Glycosylphosphatidylinositol (GPI) signal anchor sequence (SEQ ID NO:57) and C-terminal targeting signal of K-Ras4B (Ras) protein (SEQ ID NO:58).

Regarding the CaaX box motif: C is the cysteine that is prenylated, a is any aliphatic amino acid, and the identity of X determines which enzyme shall act on the protein. Farnesyltransferase recognizes CaaX boxes where X=M, S, Q, A, or C, whereas Geranylgeranyltransferase I recognizes CaaX boxes with X=L or E. A preferred protein transduction domain is the TAT protein according to SEQ ID NO:42. A preferred tag is the HA tag (SEQ ID NO: 43) or a fluorescent protein tag, such as GFP. A fusion protein according to the third aspect of the present invention is also preferably neuroprotective.

In a fourth aspect the present invention relates to a nucleic acid encoding one or more inventive polypeptides of the present invention according to the first aspect, the second aspect and/or one or more fusion proteins according to the third aspect of the invention. The inventive nucleic acid may take all forms conceivable for a nucleic acid. In particular the nucleic acids according to the present invention may be RNA, DNA or hybrids thereof. They may be single-stranded or double-stranded. The may have the size of small transcripts or of entire genomes, such as a viral genome. As used herein, a nucleic acid encoding one or more inventive polypeptides of the present invention may be a nucleic acid reflecting the sense strand. Likewise, the antisense strand is also encompassed. The nucleic acid may encompass a heterologous promotor for expression of the inventive polypeptide, such as a viral promotor or bacterial promotor. It is understood that a nucleic acid according to the present invention cannot encode a full length TRPM4 gene and will preferably also not encode the sequence of the transmembrane and/or C-terminal domain of TRPM4.

In a fifth aspect the present invention relates to a vector comprising a nucleic acid according to the present invention. Such vector may for example be an expression vector allowing for expression of an inventive polypeptide. Such vector may for example be a viral expression vector. Said expression may be constitutive or inducible. The vector may also be a cloning vector comprising the sequence of the nucleic acid of the current invention for cloning purposes.

In a sixth aspect the present invention relates to (preferably isolated) cells comprising a polypeptide, fusion protein, nucleic acid, and/or vector according to the present invention. The cells may be selected in particular from the group consisting of bacterial cells and yeast cells (e.g. for production purposes) as well as mammalian cells (e.g. for therapeutic purposes, but possibly also for production purposes).

In seventh aspect the present invention relates to a non-human animal, in particular a non-human mammal, comprising a polypeptide, fusion protein, nucleic acid, vector and/or cell according to the present invention. Such animal may for example be selected from the group consisting of mouse, rat, dog, cat, cow, monkey, horse, hamster, guinea pig, pig, sheep, goat, rabbit etc. If the respective polypeptide can be adequately expressed, such animal will be better protected against NMDA receptor induced cytotoxicity and may better withstand neurological complications than their counterparts without such nucleic acid. Moreover, such animals could also be used to study in more detail the mechanisms involved in extrasynaptical toxic NMDA receptor signaling.

In eighth aspect the present invention relates to a composition comprising a polypeptide, fusion protein, nucleic acid, vector and/or cell according to the present invention, and further comprising a pharmaceutically acceptable carrier, diluent or excipient. In a preferred embodiment, the composition comprises a nanoparticle comprising said polypeptide, fusion protein, nucleic acid, vector and/or cell according to the present invention. The nanoparticle may be designed to release said polypeptide, fusion protein, nucleic acid, vector and/or cell over time.

In a ninth aspect the present invention relates to a compound for use in a method of treating or preventing a disease of the human or animal body, wherein the compound is selected from the group consisting of:
i) a polypeptide according to the first aspect of the present invention,
ii) a polypeptide according to the second aspect of the present invention,
iii) a fusion protein according to the third aspect of the present invention,
iv) a nucleic acid according to the fourth aspect of the present invention,
v) a non-polypeptide compound binding to SEQ ID NO:3 or its derivative as defined in the first aspect of the invention,
vi) a composition according to the eighth aspect of the present invention,
vii) a compound of general formula I:

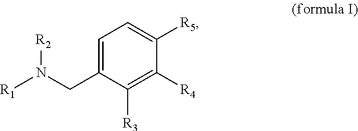

wherein:
   R₁ and R₂ are each independently selected from hydrogen, alkyl_{(C≤12)}, and substituted alkyl(c12); and
   R₃, R₄ and R₅ are each independently selected from hydrogen, hydroxy and halo; or
a pharmaceutically acceptable salt, solvate, polymorph, tautomer, racemate, or enantiomer thereof; or
viii) a compound selected from the group of compounds consisting of:

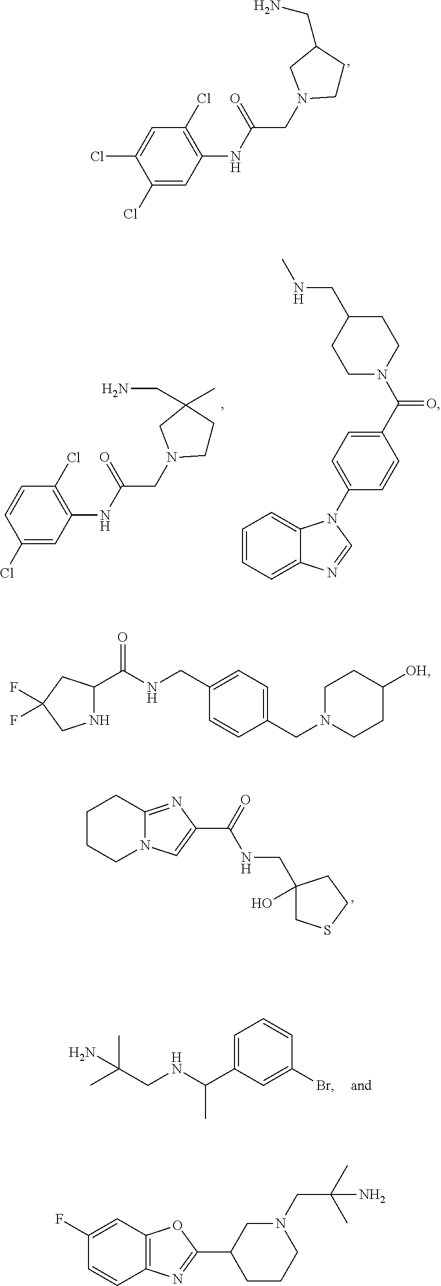

and a pharmaceutically acceptable salt, solvate, polymorph, tautomer, racemate or enantiomer of any of these compounds.

As mentioned above, the compound for use according to the ninth aspect may be a compound according to general formula I. According to said formula, R₁ and R₂ are each independently selected from hydrogen, alkyl_{(C≤12)}, and substituted alkyl_{(C≤12)}. The term “alkyl”, when used without the “substituted” modifier, refers to a monovalent saturated aliphatic group with a carbon atom as the point of attachment, a linear or branched acyclic structure, and no atoms other than carbon and hydrogen. Preferably, the alkyl is linear. The groups —CH₃ (Me), —CH₂CH₃(Et), —CH₂CH₂CH₃ (n Pr or propyl), —CH(CH₃)₂ (i Pr, iPr or isopropyl), —CH₂CH₂CH₂CH₃ (n Bu), —CH(CH₃)CH₂CH₃ (sec-butyl), —CH₂CH(CH₃)₂ (isobutyl), —C(CH₃)₃ (tert-butyl, t butyl, t Bu or tBu), and —CH₂C(CH₃)₃ (neo-pentyl) are non-limiting examples of alkyl groups. When alkyl is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Preferably, the one or more hydrogen atom has been replaced with —NH₂ or —OH, even more preferably —NH₂. Preferably, only one hydrogen atom has been replaced. Most preferably, only one hydrogen atom at a terminal carbon atom has been replaced. Preferably, R₁ and/or R₂ are alkyl_{(C≤12)}, and substituted alkyl_{(C≤12)}. Even more preferably R₁ and/or R₂ are selected from alkyl_{(C≤6)}, and substituted alkyl_{(C≤6)}. Even more preferably R₁ and/or R₂ are selected from alkyl_{(C≤4)}, and substituted alkyl_{(C≤4)}. Preferably, one of R₁ and R₂ is alkyl, while the other is selected from substituted alkyl. More preferably, R₁ is —CH₂CH₂NH₂. More preferably, R₂ is a linear alkyl_{(C≤4)} or —CH₂CH₂OH. Most preferably, R₁ is —CH₂CH₂NH₂ and R₂ is a linear alkyl_{(C≤4)}.

Furthermore, according to general formula I, R₃, R₄ and R₅ are each independently selected from hydrogen, hydroxy and halo. Preferably, R₃, R₄ and R₅ are selected from hydrogen and halo. Preferably, one, more preferably two of R₃, R₄ and R₅ are hydrogen. Preferably, R₅ is hydrogen. Preferably, only one of R₃, R₄ and R₅ is halo. More preferably R₃ or R₄ is halo. More preferably, R₃ or R₄ is selected from Cl, Br, and I. Even more preferably, R₃ or R₄ is selected from Cl and Br. Most preferably, R₃ or R₄ is Cl.

Preferred compounds according to general formula I are:

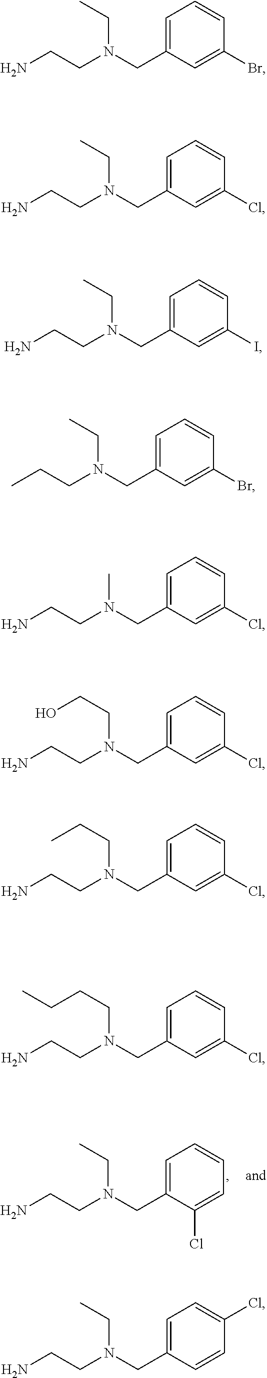

as well as any pharmaceutically acceptable salt, solvate, polymorph, tautomer, racemate or enantiomer of any of these compounds. Most preferred is the compound according to the following formula

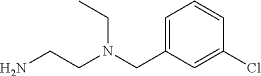

as well as any pharmaceutically acceptable salt thereof.

Wherever herein specific chemical formula are provided, the respective charged/protonated forms of said formula are also specifically contemplated as being disclosed herein and as being useful for carrying out the present invention. Preferably, any amino residue of such formula is protonated and thus positively charged.

Preferably, the disease (to be treated according the ninth aspect of the invention) is treated or prevented by inhibiting NMDA receptor mediated cytotoxicity, in particular by inhibiting NMDA receptor/TRPM4 complex formation.

In a tenth aspect the present invention relates to a method of treating or preventing a disease of the human or animal body, the method comprising administering an effective amount of a compound to a subject in need of treatment or prevention of the disease, wherein the compound is selected from the group consisting of:
i) a polypeptide according to the first aspect of the present invention,
ii) a polypeptide according to the second aspect of the present invention,
iii) a fusion protein according to the third aspect of the present invention,
iv) a nucleic acid according to the fourth aspect of the present invention,
v) a non-polypeptide compound binding to SEQ ID NO:3 or its derivative as defined in the first aspect of the invention,
vi) a composition according to the eighth aspect of the present invention,
vii) a compound of general formula I:

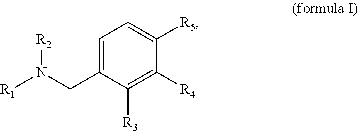

wherein:
   R₁ and R₂ are each independently selected from hydrogen, alkyl_{(C≤12)}, and substituted alkyl_{(C≤12)}; and
   R₃, R₄ and R₅ are each independently selected from hydrogen, hydroxy and halo; or
a pharmaceutically acceptable salt, solvate, polymorph, tautomer, racemate, or enantiomer thereof; or
viii) a compound selected from the group of compounds consisting of:

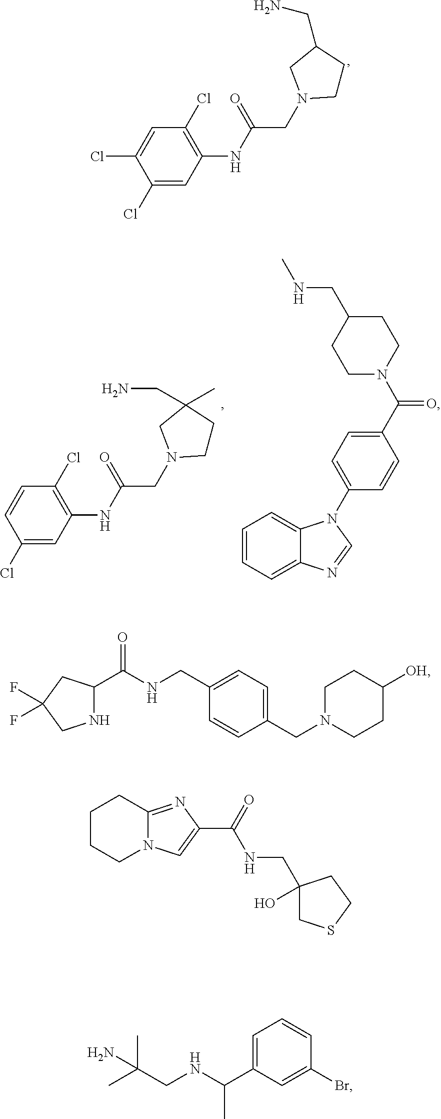 [SMILES: NCC1CCN(CC(=O)Nc2cc(Cl)c(Cl)cc2Cl)C1 ; CC1(CN)CCN(CC(=O)Nc2cc(Cl)ccc2Cl)C1 ; CNCC1CCN(C(=O)c2ccc(-n3cnc4ccccc43)cc2)CC1 ; O=C(NCc1ccc(CN2CCC(O)CC2)cc1)C1CC(F)(F)CN1 ; O=C(NCC1(O)CCSC1)c1cn2c(n1)CCCC2 ; CC(NCC(C)(C)N)c1cccc(Br)c1]

and
and a pharmaceutically acceptable salt, solvate, polymorph, tautomer, racemate or enantiomer of any of these compounds.

If the compound to be administered according to the tenth aspect of the invention is a compound according to general formula I, then the same respective embodiments and preferences as set out above for the ninth aspect are specifically considered. In particular, the compound according to the following formula

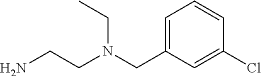

as well as any pharmaceutically acceptable salt thereof is a preferred embodiment for carrying out the tenth aspect of the invention.

The method in the context of the tenth aspect of the invention may be a method for inhibiting NMDA receptor mediated toxicity, wherein an effective amount of an above mentioned compound is administered to the subject, thereby inhibiting NMDA receptor mediated toxicity.

The disease in the context of the ninth aspect or tenth aspect of the invention is preferably a neurological disease, in particular a neurodegenerative disease, or diseases potentially leading to or involving neurodegenerative events, for example infections leading to neurodegenerative events, in particular in the brain. The neurological or neurodegenerative disease may in some embodiments have an inflammatory component, i.e., is a neuroinflammatory disease. The neurodegenerative disease may by a progressive neurodegenerative disease. Preferably, the disease is selected from the group consisting of stroke, Alzheimer's disease (AD), amyotrophic lateral sclerosis (ALS), Huntington's disease (HD), traumatic brain injury, multiple sclerosis, glutamate induced excitotoxicity, dystonia, epilepsy, optic nerve disease, diabetic retinopathy, glaucoma, pain, particularly neuropathic pain, anti-NMDA receptor encephalitis, viral encephalopathy, vascular dementia, microangiopathy, Binswanger's disease, cerebral ischemia, hypoxia, Parkinson's disease, schizophrenia, depression, cerebral malaria, toxoplasmosis (due to the risk of toxoplasmosis-associated brain damage), HIV infection/AIDS (due to the risk of HIV)-associated brain damage, and Zika virus infection (due to the possibility of Zika virus-associated brain damage), or any other viral infection potentially leading to neurodegenerative events and corresponding neuronal or brain damage, respectively. In a further embodiment the disease may be a brain tumour, in particular a glioblastoma. Three papers published recently in Nature (see Nature, 2019, Vol 573 pages 499-501) show that glioblastoma cells express NMDA receptors and that their growth is enhanced/stimulated by the activation of NMDA receptors.

Therefore, the growth of glioblastoma cells may be inhibited when NMDA receptor signaling is blocked, e.g. by compounds as described herein. On contrast thereto, conventional blockers of NMDA receptors cannot be used in this case because they interfere with the physiological role of NMDA receptors in normal synaptic transmission and cognitive functions such as memory.

The compound for use according to the ninth aspect or used in the method of the tenth aspect of the invention may be a polypeptide according to the first aspect of the present invention. The inventors have found that polypeptides comprising the respective TRPM4 fragment (i.e. SEQ ID NO:3 or a derivative thereof) can be used to protect against NMDA receptor induced excitotoxicity. Such a polypeptide can for example be administered in an effective amount to a patient suffering from neurological and/or neurodegenerative disease. Such polypeptide could for example be administered directly to the subject. In the alternative, a vector encoding such polypeptide could be used to express the polypeptide in the cells of the subject. The same considerations apply if the compound is polypeptide according to the second aspect of the present invention, e.g. an antibody or anticalin, or a fusion protein according to the third aspect of the invention. If the compound is a fusion protein according to the present invention, then it is particularly preferred if the fusion protein comprises means to direct the fusion protein towards the cell membrane, in particular towards the cytosolic side of the membrane, as this is where the N-terminal domain of TRPM4 is typically found in a cell. A similar effect is attained if the fusion protein comprises a protein transduction domain allowing the polypeptide to pass from without through the cell membrane and enter the cytosol of the cell. In cases where the compound is a nucleic acid according to the fourth aspect of the present invention, such nucleic acid may also be used in the context of a gene therapy, for example if is inserted permanently or temporarily in the genome of the subject to be treated. In cases where the compound is a vector according to the fifth aspect of the present invention, such vector is preferably a viral vector. The compound may also be a non-polypeptide compound binding to SEQ ID NO:3 or its derivative as defined in the first aspect of the invention, for example a corresponding DNA aptamer or a small molecule.

Typically, the method of treatment (of the ninth aspect or tenth aspect) will focus on stopping or slowing down the progression of the disorder. In the alternative, such compound can also be administered in a preventive manner, e.g. in situations where the subject is at (an increased) risk of suffering from a neurological and/or neurodegenerative disease. This includes an acute (increase in) risk (e.g. a thrombotic stroke after surgery) as well as a continuous risk (e.g. due to a genetic and/or familial predisposition for a given neurological and/or neurodegenerative disorder).

The subject to be treated is preferably a mammal, preferably selected from the group consisting of human, mouse, rat, dog, cat, cow, monkey, horse, hamster, and guinea pig, pig, sheep, goat, rabbit etc. Most preferably, the subject is a human being. If the compound for use according to the ninth aspect or used in the method of the tenth aspect of the invention is a polypeptide according to the first aspect of the invention, a fusion protein according to the third aspect of the invention, or a respective nucleic acid or vector encoding the same, then preferably said compound matches the subject to be treated. For example, for treatment of a human being the polypeptide according to the first aspect of the present invention will preferably comprise the human sequence, i.e. an amino acid sequence according to SEQ ID NO:3. In contrast, for treatment of mice, the polypeptide according to the present invention would preferably comprise an amino acid sequence according to SEQ ID NO:5, etc.

For the purposes of the ninth aspect and tenth aspect of the invention, the person skilled in the art will be readily capable of selecting an appropriate route of administration, depending on the specific disease to be treated or prevented and/or body part to be treated. The route of administration may be for example oral, topical, intranasal, parenteral, intravenous, rectal or any other route of administration suitable in the specific context. For example, if the disease is a cerebrovascular disease, e.g. stroke, then intranasal administration is a preferred route of administration. Intranasal administration is known to the skilled person as being particularly suitable for administering neuroprotective compounds in general, for example in the context of treatment of stroke and stroke induced brain damage.

The compound may be administered in all suitable forms for the given purpose, including for example tablet, capsule, granule, powder, liquid, ointment, lotion, cream, spray, inhalant and the like. The compound may be formulated for being administered parenterally, e.g. by intravenous injection or intravenous infusion. In a particularly preferred embodiment, the compound for use according to the ninth aspect or used in the method of the tenth aspect of the invention may be formulated for being administered intranasally, for example as ointment or cream, or as, e.g., saline solution to be applied via a spray to the nose. The compound for use according to the ninth aspect or used in the method of the tenth aspect of the invention may also be formulated for retarded or sustained release and/or encapsulated in nanoparticles or vesicles.

In an eleventh aspect the present invention relates to the use of a polypeptide or fusion protein according to the first, second or third aspect of the invention, respectively, in a protein-protein interaction assay. Preferably, the protein-protein interaction assay is an in vitro protein-protein interaction assay. It is contemplated that a polypeptide/fusion protein according the first, second or third aspect of the invention will be particularly useful for identifying further binding partners of TRPM4 proteins. The protein-protein interaction under scrutiny in such assays is preferably an interaction within the region specified by the amino acid sequence according to SEQ ID NO:3, or its derivative sequence, as defined above for the first, second and third aspect of the invention. Such binding partners may turn out to be neurotoxic, neuroprotective or neither thereof. In this context the polypeptide/fusion protein will not only provide insights regarding its own interaction partners but will also shed light on interactions of other compounds involved in TRPM4 signaling, for example if certain complexes can no longer be formed due to (e.g., competitive) inhibition. The person skilled in the art is familiar with a large number of possible assays for determining protein-protein interactions, including biochemical, biophysical and genetic methods. Non-limiting examples are immunoprecipitation, bimolecular fluorescence complementation (e.g. split-TEV, split-GFP), affinity electrophoreses, immunoelectrophoresis, phage display, tandem affinity purification, chemical crosslinking followed by mass spectrometry analysis, surface plasmon resonance, fluorescence resonance energy transfer, nuclear magnetic resonance imaging, etc. The protein-protein interaction assay may be an in vitro, ex vivo or an in vivo assay. Most preferably, the protein-protein interaction assay is an in vitro assay. However, for example in the context of live imaging such assay may also be an in vivo assay. In cases where the assay is an in vivo assay, it is preferably not an assay in a human being.

In a twelfth aspect, and in a similar context as the eleventh aspect, the present invention relates also to a method for identifying a compound potentially interacting with a TRPM4 protein comprising the amino acid sequence of a polypeptide according to the first aspect of the invention, wherein the method comprises:
i) computer-assisted virtual docking of a candidate compound to an amino acid sequence according to SEQ ID NO:3, or a derivative of said sequence, wherein said amino acid sequence is present in a virtual 3D structure of a polypeptide comprising said amino acid sequence, and
ii) determining the docking score and/or internal strain for docking the candidate compound virtually to the amino acid sequence according to SEQ ID NO:3, or its derivative, and optionally
iii) contacting in vitro or in vivo the candidate compound with a TRPM4 protein to determine whether the candidate compound modulates the activity of said TRPM4 protein or not.

Methods for in silico docking of candidate compounds to protein structures are well known in the art. The candidate compound can be any compound. Typically, the compound will be a small molecule. Preferably, the small molecule is not ATP. More preferably, the small compound is not a nucleotide at all and/or does not comprise an adenosine moiety. It is also possible that the compound is a large biomolecule, such as an antibody or the like. Collections of compounds are for example available from Schrödinger LLC (New York, NY, USA). The 3D structure can be any structure comprising an amino acid sequence according to SEQ ID NO:3, or a derivative of said sequence. The 3D structure can be a 3D structure of human TRPM4 or of parts thereof. The derivative is as defined above, e.g. for the first aspect of the invention. Preferably, the derivative is a sequence having at least 80% sequence identity with SEQ ID NO:3, or ii) a sequence according to SEQ ID NO:4. In the present case, various structures of TRPM4 proteins are available to the skilled person, for example in the Protein Data Bank, and can be used for the method of the twelfth aspect. Without being limited thereto, suitable 3D structures for such method are the structures of human TRPM4, e.g. 5WP6, 6BQR, 6BQV etc., or the mouse structure 6BCO.

The 3D structure may be based for example on a structured obtained by x-ray crystallography analysis, NMR spectroscopy analysis, cryo-EM, or derived from homology modeling. It is understood that the method according to the twelfth aspect of the invention will encompass docking of a candidate compound to the region in the TRPM4 structure, which corresponds to the amino acid sequence according to SEQ ID NO:3 or its derivative, and does not encompass docking to regions of the structure, which do not relate to the the amino acid sequence according to SEQ ID NO:3 or its derivative. However, if docking to the region with SEQ ID NO:3 or its derivative requires in parallel interaction with other amino acid residues outside said region, than such docking is also encompassed by the method according to the twelfth aspect of the invention. Docking itself can be accomplished by a variety of methods known to the person skilled in the art and respective software is publicly available (see for example Schrödinger LLC, New York, NY, USA). A respective analysis can also be ordered from commercial providers, for example Proteros biostructures GmbH, Planegg, Germany). The method of the twelfth aspect of the present invention may also be used to identify inhibitors of NMDA receptor mediated excitotoxicity.

In a thirteenth aspect, the present invention relates to a compound for use in a method of treating or preventing a disease of the human or animal body, wherein the compound is an inhibitor of NMDA receptor −TRPM4 complex formation. An inhibitor of NMDA receptor-TRPM4 complex formation can be identified by testing a given candidate compound in an assay as set out for instance in the examples of the present invention (see in particular, without being limited thereto, example 1, Methods and Materials, Examples 11, 12, and 15). An inhibitor of NMDA receptor-TRPM4 complex formation is for example any of the compounds discussed in the context of the ninth aspect of the invention. All embodiments disclosed in this context are specifically contemplated also for the thirteenth aspect of the invention. Preferably, the inhibitor of NMDA receptor-TRPM4 complex formation does not block the NMDA receptor channel per se (for a corresponding test see Example 14). Preferably, the inhibitor of NMDA receptor-TRPM4 complex formation does not block the TRPM4 channel per se (for a corresponding test see Example 17). The disease may be any disease as already discussed in the context of the ninth or tenth aspect of the invention.

In a fourteenth aspect, the present invention relates to a method of treating or preventing a disease of the human or animal body, the method comprising administering an effective amount of a compound to a subject in need of treatment or prevention of the disease, wherein the compound is an inhibitor of NMDA receptor −TRPM4 complex formation. Regarding the inhibitor of NMDA receptor −TRPM4 complex formation and disease, reference is made to the thirteenth aspect of the invention and the ninth and tenth aspect of the invention, respectively. All embodiments disclosed in this context are specifically contemplated also for the fourteenth aspect of the invention.

In a fifthteenth aspect, the present invention relates to a cell, in particular a non-neuronal cell such as a HEK293 cell, wherein said cell expresses a recombinant NMDA receptor, and wherein expression of TRPM4 is absent, knocked down or knocked-out, preferably knocked-out. The cell is preferably an isolated cell, i.e. not residing in a human or animal body. The cell is preferably not expressing any glutamate receptors or subunits. The cell is preferably a mammalian cell, such as a human cell. Preferably, the cell can be cultured as cell line. Such cell is perfectly suited to study NMDA receptor activity, be it now inhibition or activation or modulation. Such studies may be pharmacological studies aimed at discovery and/or characterization of new compounds (small molecules, peptides, proteins etc.) and known compounds (small molecules, peptides, proteins etc.) that block or enhance one or several aspects of NMDA receptor functions, which include but are not limited to ion conductance, activation and de-activation kinetics, and magnesium block and magnesium unblock. Such studies may also include assessments of NMDA receptor structure-function relationships in which plasmids are being transfected that contain expression vectors for the various subunits of NMDA receptors (GRIN1, GRIN2A, GRIN2B, or other GRIN2 subunits or GRIN3) that contain point mutation or deletion mutants followed by functional analysis of parameters such as those mentioned above (ion conductance etc.).

Conventional approaches suffer from the drawback that expression of recombinant NMDA receptor (for example in HEK 293 cells) typically leads to cell toxicity and death. Therefore, conventional approaches need to grow such cells in presence of inhibitors NMDA receptors, which makes studies of NMDA receptors, and interpretation of the results, in those cells difficult and complicated. By decoupling NMDA receptor activity from interaction with TRPM4 said cell toxicity and death can be prevented and thus there is no need any more for cell culture in presence of inhibitors NMDA receptors.

In a sixteenth aspect, the present invention relates to the use of (channel) inhibitors of a TRPM4 protein, for example of inhibitors of human TRPM4, for inhibiting NMDA receptor mediated excitotoxicity. The inhibitor of (e.g. human) TRPM4 can be any known TRPM4 inhibitor, such as glibenclamide, 9-phenanthrol, tolbutamide, repaglinide, nateglinide, meglitinide, midaglizole, LY397364, LY389382, glyclazide, glimepiride, estrogen, estradiol, estrone, estriol, genistein, non-steroidal estrogen, phytoestrogen, zearalenone, 5-butyl-7-chloro-6-hydroxybenzo[c]-quinolizium chloride, flufenamic acid, and spermine. Knockdown of expression of TRPM4 is also considered to be an inhibitor of TRPM4 protein. The use can occur in vitro or in vivo. If the use occurs in vivo and is of therapeutic nature, then such embodiment reflects an inhibitor of a TRPM4 protein for use in a method of treatment of a disease of the human or animal body, wherein the disease is: i) treated or prevented by inhibiting NMDA receptor mediated cytotoxicity and/or ii) caused by NMDA receptor mediated excitotoxicity.

The term “comprising”, as used herein, shall not be construed as being limited to the meaning “consisting of” (i.e. excluding the presence of additional other matter). Rather, “comprising” implies that optionally additional matter may be present. The term “comprising” encompasses as particularly envisioned embodiments falling within its scope “consisting of” (i.e. excluding the presence of additional other matter) and “comprising but not consisting of” (i.e. requiring the presence of additional other matter), with the former being more preferred.

The term “nanoparticle”, as used herein, preferably refers to a particle between 1 and 100 nanometers in size. The nanoparticle may comprise a polymer. The particle may comprise silica, in particular a silica core. The particle may comprise an outer layer with functional groups. Such functional groups may for example allow for linking the nanoparticle to a compound of interest.

### FIGURES

In the following a brief description of the appended figures will be given. The figures are intended to illustrate aspects of the present invention in more detail. However, they are not intended to limit the scope of the invention.

FIG. 1 illustrates that knockdown of TRPM4 protein by using RNA interference protects neurons from NMDA receptor mediated toxicity. Vehicle was water. shTRMP4-1 is depicted in SEQ ID NO:44, shTRMP4-2 is depicted in SEQ ID NO:45. All data are shown as mean±s.d. n=3 independent experiments. Two-Way ANOVA followed with Dunnett post hoc test. n.s. not significant. * p≤0.05, ** p≤0.01, *** p≤0.001, **** p ≤0.0001.

FIG. 2 illustrates that mouse TRPM4 contains a polypeptide element conferring protection against NMDA induced cell death. A) Neuroprotective effect of 4 different fragments of mouse TRPM4: Amino acid residues 1-346 (SEQ ID NO:46); amino acid residues 347-689 (SEQ ID NO:47); amino acid residues 690-1036 (SEQ ID NO:48); amino acid residues 1037-1213 (SEQ ID NO:49); Of these only SEQ ID NO:47 is neuroprotective. B) Neuroprotective effect of 4 different fragments of a polypeptide with the sequence according to SEQ ID NO:47: amino acid residues 347-467 (SEQ ID NO:50); amino acid residues 468-548 (SEQ ID NO:51); amino acid residues 536-648 (SEQ ID NO:52); amino acid residues 633-689 (SEQ ID NO:5). Of these only SEQ ID NO:5 is neuroprotective. All data are shown as mean±s.d. n=3 independent experiments. Two-Way ANOVA followed with Dunnett post hoc test. * p≤0.05, ** p≤0.01, *** p≤0.001.

FIG. 3 illustrates protective properties of various variants of the polypeptide with the sequence of SEQ ID NO:5. A) Analysis of NMDA induced neuronal death in neurons infected with rAAV and overexpressing SEQ ID NO:47 or SEQ ID NO:53. SEQ ID NO:53 corresponds to SEQ ID NO:47 but comprises additionally a membrane-anchor (GPI). The experiments showed that the GPI anchor increased the protective effects of the polypeptide according to SEQ ID NO:47, leading to less cell death. B) Analysis of NMDA induced neuronal death in in cultured neurons infected on DIV3 with the rAAVs expressing SEQ ID NO:5, SEQ ID NO:54 or SEQ ID NO:55 and challenged with 20 M NMDA for 10 min on DIV17, with cell death assessed 24 h later. SEQ ID NO:54 is a derivative of SEQ ID NO:5, having a conservative substitution of two Y for two F. It is only slightly less effective in reducing NMDA receptor-mediated cell toxicity than SEQ ID NO:5. In contrast, a corresponding region deriving from related but different mouse protein TRPM5, SEQ ID NO:55, sharing only about 60% sequence identity with SEQ ID NO:5, is not capable of reducing NMDA receptor-mediated cell toxicity. All data are shown as mean±s.d. n=3 independent experiments. Two-Way ANOVA followed with Dunnett post hoc test. n.s. not significant. * p≤0.05, ** p≤0.01, *** p≤0.001, **** p ≤0.0001.

FIG. 4 illustrates the effect of preexposure of neurons to 1 and 10 μg of a fusion peptide comprising the peptide of SEQ ID NO:5 and a protein transduction domain (TAT, SEQ ID NO:42). The fusion protein (SEQ ID NO:5+TAT) had the amino acid sequence according to SEQ ID NO:56. The experiments showed that the application of SEQ ID NO:56 protected neurons from NMDA excitotoxicity. Vehicle was water.

FIG. 5 illustrates infarct volumes in whole ischemic brains of mice subjected to stereotactic injection of recombinant adeno associated viruses (rAAVs) encoding SEQ ID NO:5 3 weeks before MCAO or sham surgery. PBS and GFP were used as control. The infarct size was determined 7 days after MCAO (mean±SD). Statistical analysis was determined by t-test; statistically significant differences are indicated with asterisks (n=5-8). **P<0.005, ***P<0.001.

FIG. 6 illustrates the preventive effect of polypeptides according to SEQ ID NO:5 and SEQ ID NO:54 against neuronal mitochondrial membrane potential break down in primary mouse hippocampal neurons. Addition of carbonylcyanide-p-trifluoromethoxyphenylhydrazone (FCCP) leads to breakdown of the mitochondrial membrane potential. A) Breakdown of the mitochondrial membrane in untransfected primary mouse hippocampal neurons; B) Delay of breakdown of mitochondrial membrane potential in presence of SEQ ID NO:5. Addition of the uncoupler FCCP at the end of the experiment leads to breakdown of the mitochondrial membrane potential; C) Comparison of the protective effect of three different polypeptides against neuronal mitochondrial membrane potential breakdown: Uni: Uninfected (negative control); SEQ ID NO:5, SEQ ID NO: 54 and SEQ ID NO: 55 (negative control). One-Way ANOVA followed with Tukey's post hoc test. n.s. not significant. **** p<0.0001.

FIG. 7 illustrates that the effects observed for SEQ ID NO:5 and SEQ ID NO: 54 are not affecting synaptic NMDA receptor signaling. In particular, synaptic NMDA receptor activation, which is boosted by the GABAA receptor antagonist Gabazine, mediated calcium influx into mitochondria are not affected by SEQ ID NO:5 and SEQ ID NO: 54. All data are shown as mean±s.d. n=10-12 from 3 independent experiments. One-Way ANOVA followed with Tukey's post hoc test. n.s. not significant. **** p≤0.0001.

FIG. 8 illustrates the neuroprotective effect of compounds identified in a virtual screen as potentially interacting with SEQ ID NO:5 in mouse TRPM4. DMSO was used as negative control. Glibenclamide, a TRPM4 inhibitor, was used as positive control. A) baseline level of cell death in HEK293 cells without induction of NMDA receptor mediated excitotoxicity; B) cell death mediated by GRIN1+GRIN2 Å receptor complexes; C) cell death mediated by GRIN1+GRIN2B receptor complexes. All data are shown as mean±s.d. n=3 independent experiments. One way ANOVA followed with Tukey's post hoc test. n.s. not significant. * p≤0.05, ** p≤0.01, *** p≤0.001, **** p≤0.0001.

FIG. 9 illustrates the neuroprotective effect of compound P4 and compound P15 against neuronal mitochondrial membrane potential breakdown in primary mouse hippocampal neurons. 30 min prior to recording, P4 and P15 were applied to cultured neurons. After a 1 min recording of baseline, excitotoxicity leading to mitochondrial membrane potential breakdown was induced with bath application of 20 M NMDA. After 10 minutes, the mitochondrial uncoupler, carbonylcyanide-p-trifluoromethoxyphenylhydrazone (FCCP) was added. Addition of FCCP leads to breakdown of the mitochondrial membrane potential. MK-801, a non-competitive, general NMDA receptor inhibitor, was used as positive control. A) Delay of breakdown of mitochondrial membrane potential in presence of DMSO, P4, P15 or MK-801; B) Quantitative comparison of protective effect of DMSO, P4 and P15 against neuronal mitochondrial membrane potential break down. The results showed that P4 and P15 were able to significantly protect mitochondrial membrane potential from NMDA excitotoxicity, respectively. All data are shown as mean±s.d. n=6 from 2 independent experiments. One-way ANOVA followed with Tukey's post hoc test. n.s. not significant. * p≤0.05, ** p≤0.01, *** p≤0.001, **** p ≤0.0001.

FIG. 10 illustrates the neuroprotective effect of compound P4 and derivatives of compound P4 (compounds 401 to 409) against NMDA excitotoxicity in primary mouse hippocampal neurons. Neurons were pre-treated for 30 min with 10 M of the indicated compound and then challenged with NMDA (20 μM) for 10 min (Transient NMDA toxicity, FIG. 10A) or with NMDA (20 μM) for 24 hours (Chronic NMDA toxicity, FIG. 10B). Cell death was assessed 24 hours after the NMDA challenge. For assessment of cell death, neurons were fixed with 4% paraformaldehyde, 4% sucrose in PBS for 15 min, washed with PBS, and counterstained with Hoechst 33258 (1 g/ml) for 10 min. The cells were mounted in Mowiol 4-88 and examined by fluorescence microscopy. The dead neurons were identified by amorphous or shrunken nuclei. All data are shown as mean±s.d. n=3-5 from 2-5 independent experiments. One-way ANOVA followed with Dunnett's post hoc test, vs vehicle group. * p≤0.05, *** p≤0.001, **** p≤0.0001.

FIG. 11 illustrates the capacity of GRIN2 Å and GRIN2B to induce—in presence of GRIN1—cell death in wild type HEK293 cells (A) and TRPM4 knock-out HEK293 cells (B) at the indicated time points after transfection.

FIG. 12 illustrates the effects of compound P4, compound P15 or MK-801 on NMDA-induced calcium influx during a 6 min NMDA (20 μM) application. Provided is a quantitative analysis of baseline (FIG. 12A), amplitude (FIG. 12B), and area under the curve (AUC, FIG. 12C). Unlike the classical NMDA receptor blocker, MK-801, which completely blocked NMDA-induced calcium transients, neither compound P4 nor P15 reduced NMDA-induced calcium transients in hippocampal neurons. The compounds do thus not affect NMDA induced calcium influx per se.

FIG. 13 provides a quantitative analysis of the ratios of GRIN2B and TRPM4 obtained by co-immunoprecipitation of the NMDA receptor/TRPM4 death complex from cortical lysates obtained from control mice and from mice 2 h, 6 h and 24 h following intraperitoneal injection of compound P4 (40 mg/kg). The NMDA receptor/TRPM4 complex was immunoprecipitated with an anti-TRPM4 antibody. A reduction in NMDA receptor/TRPM4 complex formation of 51% at 2 h and 61% at 6 h after a single intraperitoneal (i.p.) injection of 40 mg/kg of compound P4 occurs, thereby demonstrating that compound P4 effectively interfered with such complex formation. 24 h after i.p. injection of compound P4 the NMDA receptor/TRPM4 complex had reformed.

FIG. 14 provides a quantitative analysis of Brn3a-positive retinal ganglion cell (RGC) degeneration after intravitreal injection of mice with NMDA (20 nmol). The analysis is based on whole-mount retinas stained with antibodies to Brn3a 1 week after intravitreal injection of NMDA to mark live RGCs in mice receiving vehicle or compound P4. All data shown as mean±s.d. Compound P4 reduces retinal ganglion cell (RGC) degeneration after intravitreal injection of mice with NMDA (20 nmol).

FIG. 15 illustrates the effects of compound P4 and compound P15 on TRPM4 channel function in the prostate cancer cell line PC3. TRPM4 currents are characterized by their calcium dependence and outward rectification. Summary histogram shows the parameters for individual cells patched with either 0 or 10 μM free Ca²⁺ in the intracellular solution and pre-incubated at 37° C. for 30-60 min and recorded in either control solution or 10 μM P4 or P15. The results indicate that 10 μM Ca²⁺ activates a TRPM4-like outwardly rectifying current in PC3 cells and this current is not affected by P4 or P15. Data shown as mean±s.d. n=10-17 per group from 3 independent experiments. n.s. not significant.

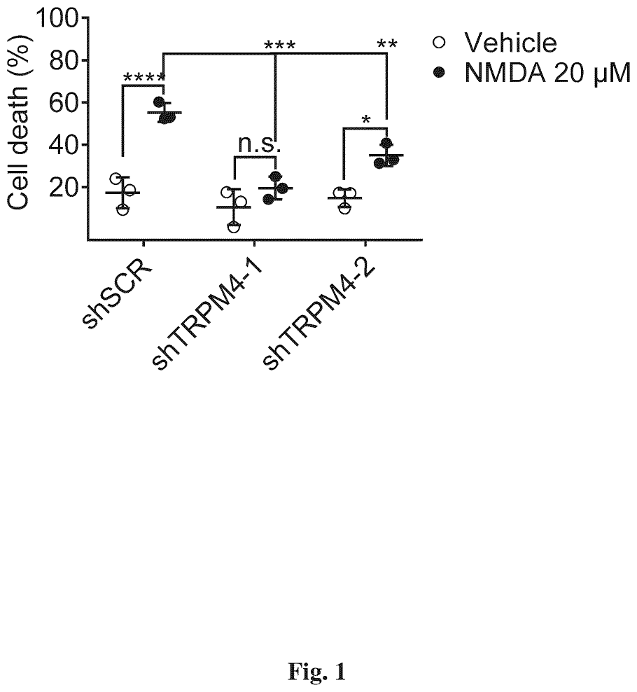

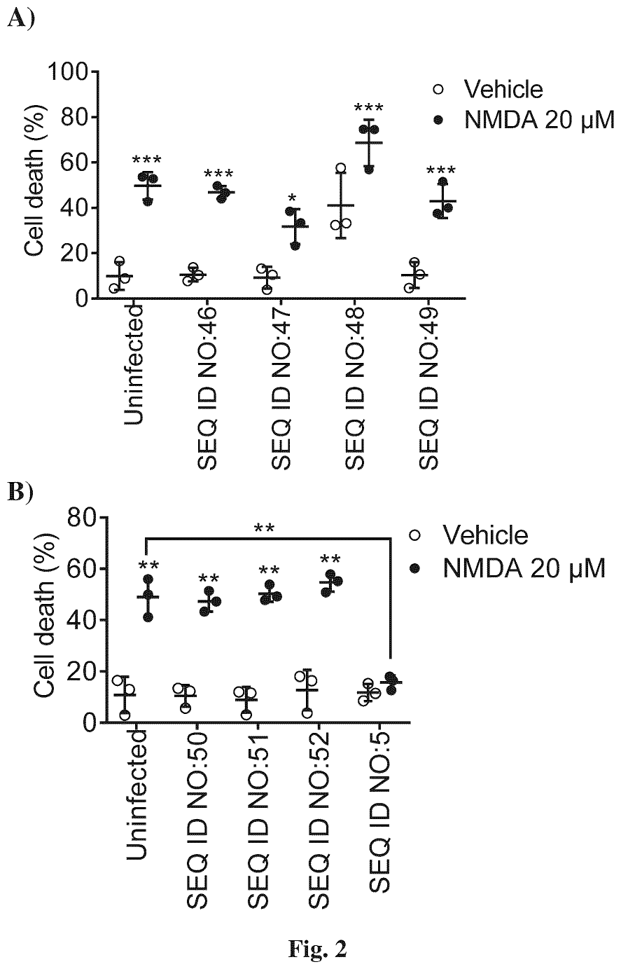

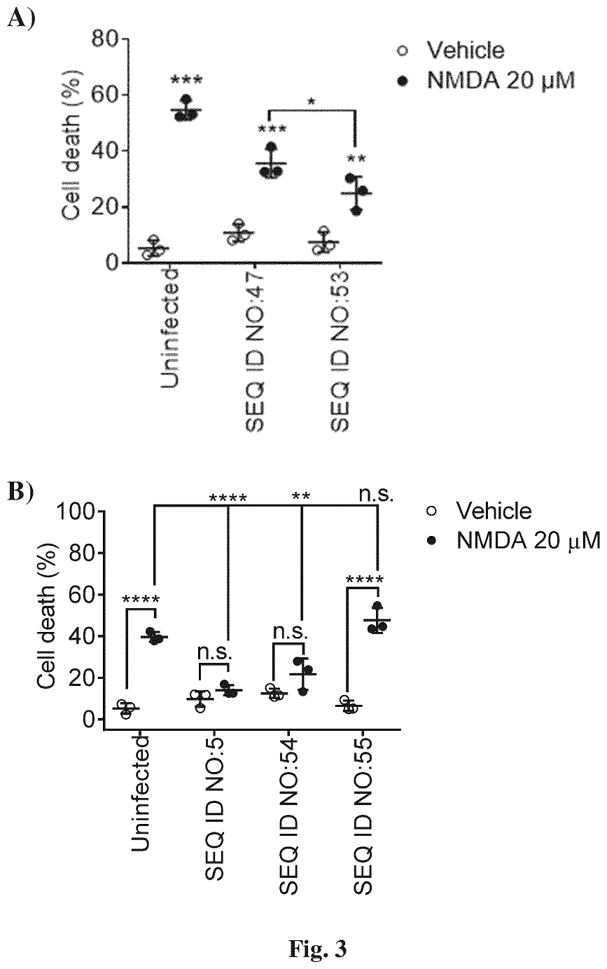

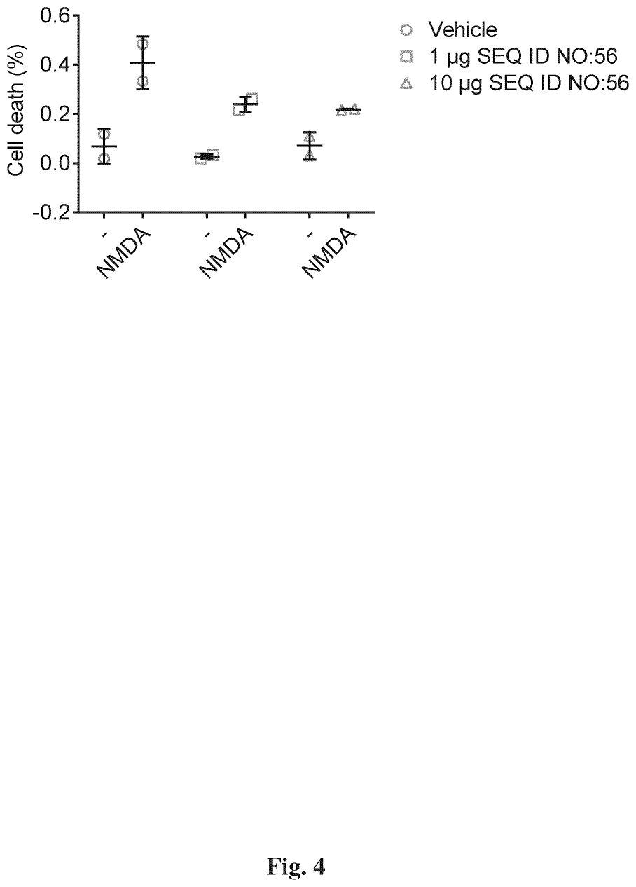

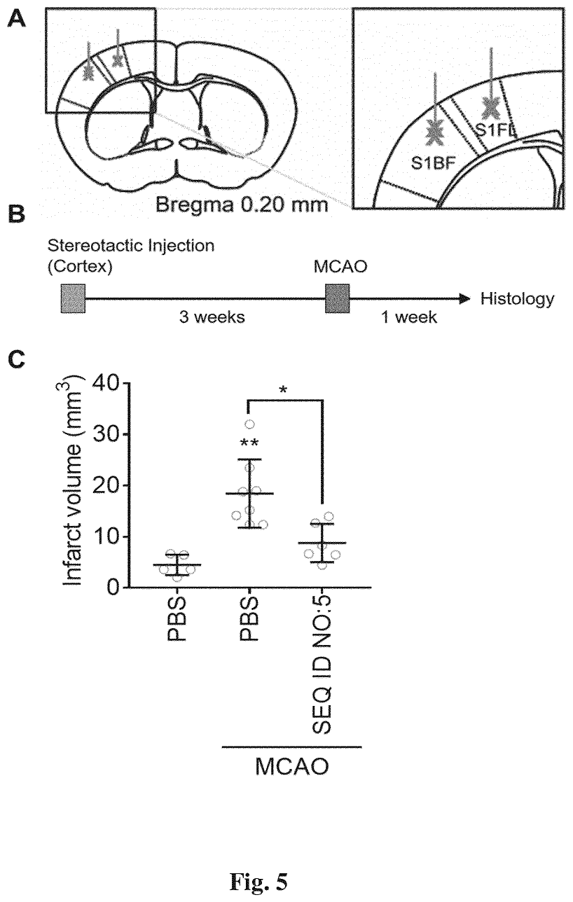

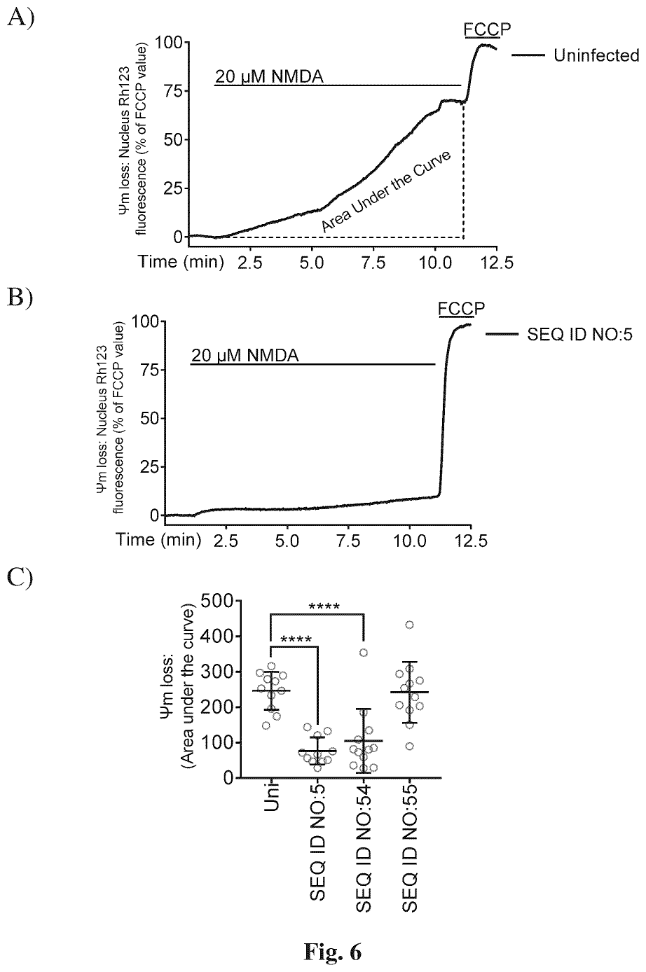

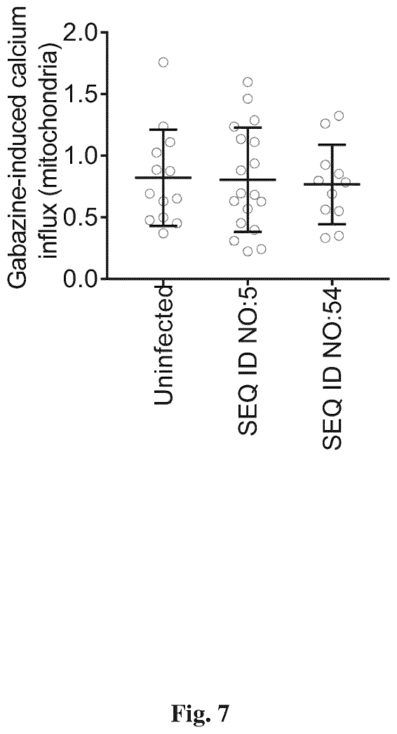

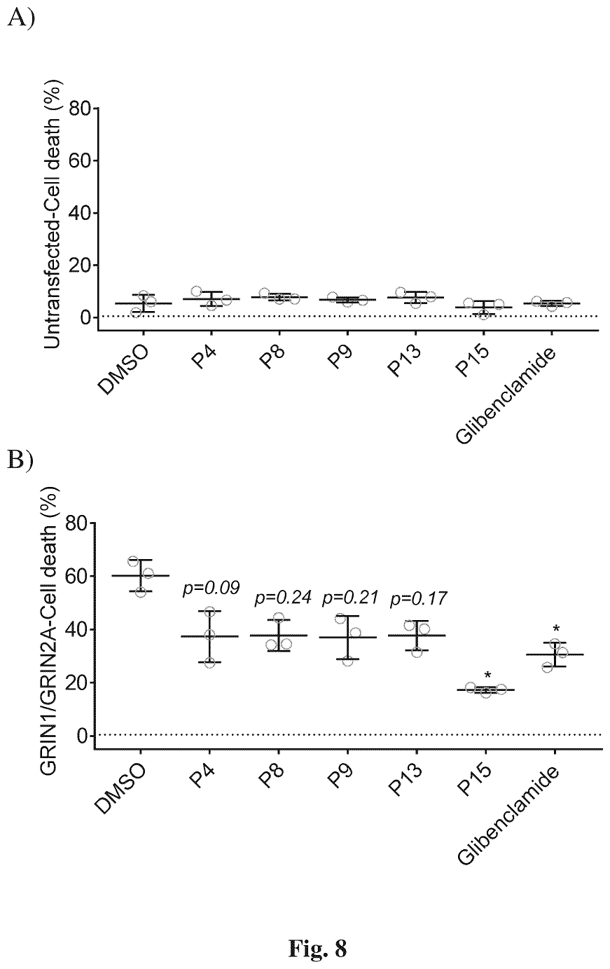

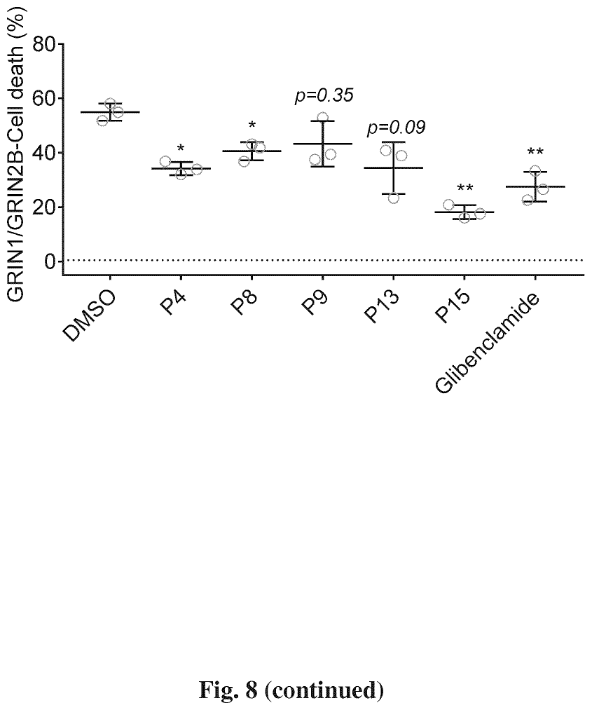

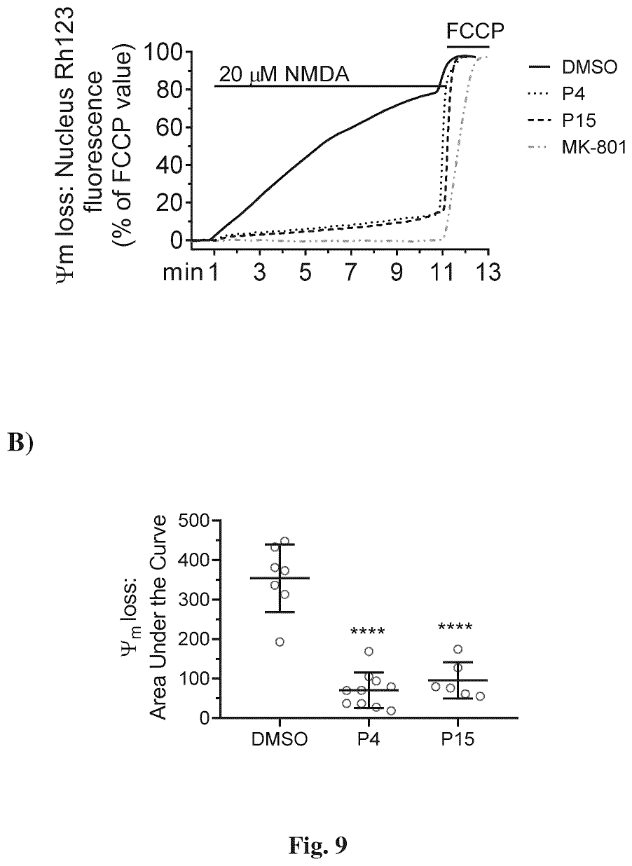

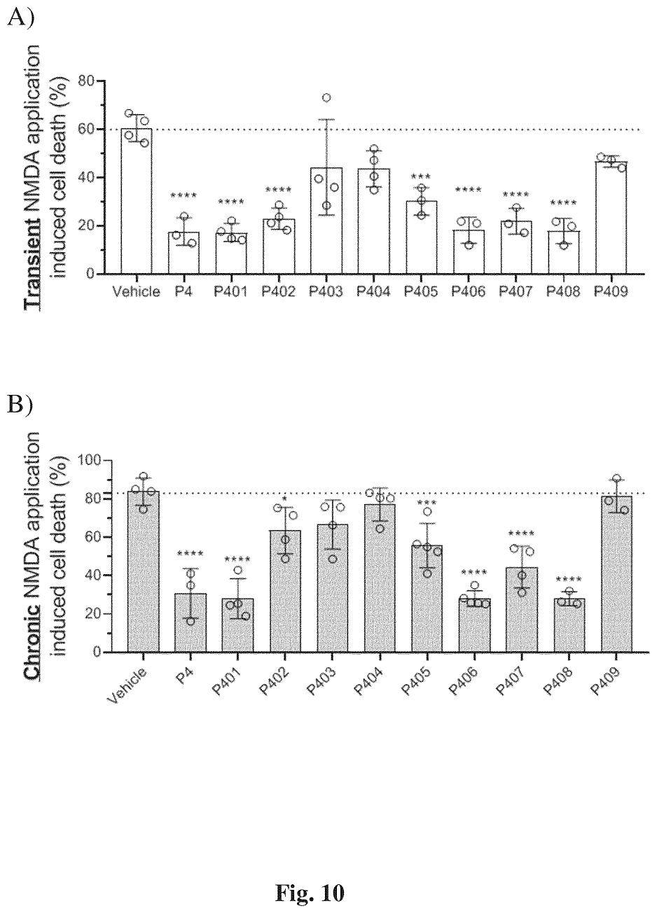

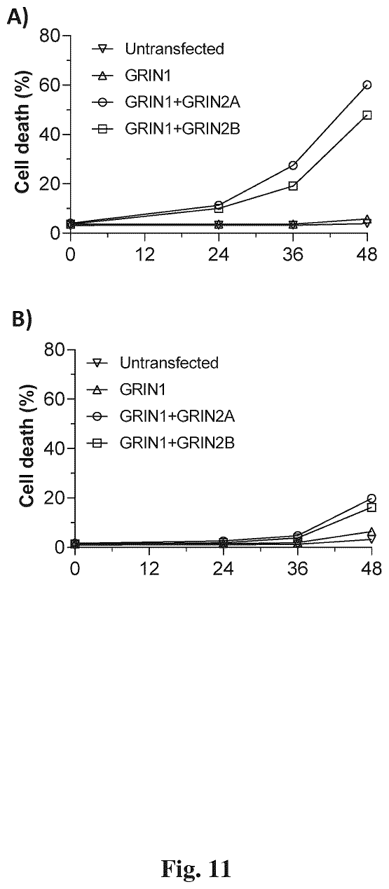

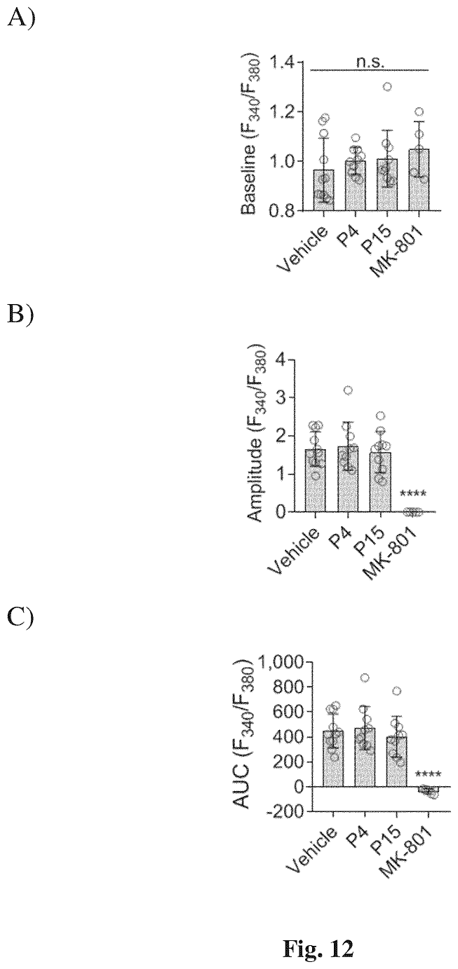

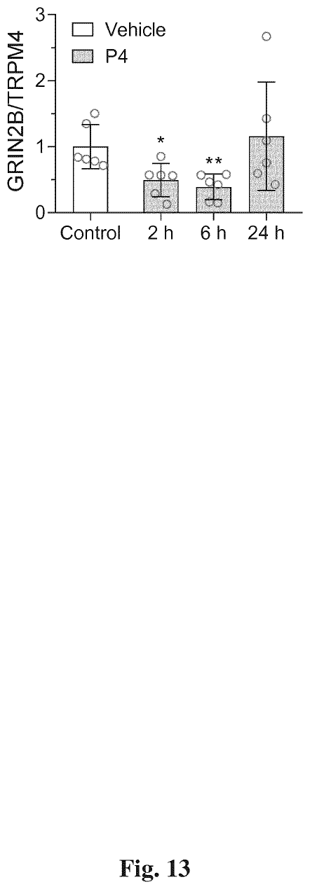

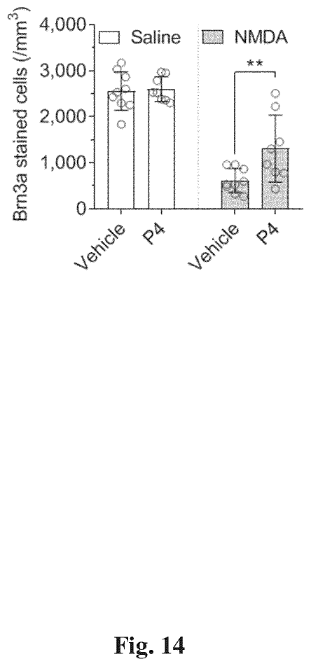

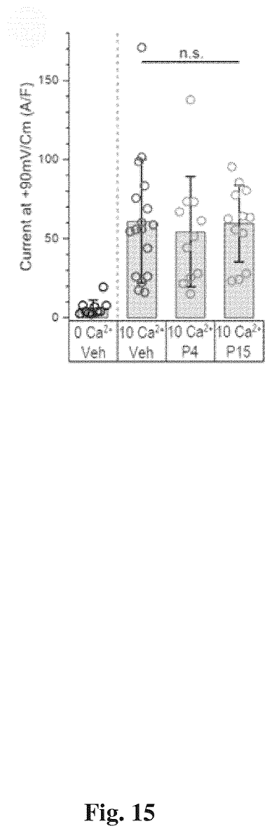

### EXAMPLES

In the following specific examples illustrating embodiments and aspects of the invention are presented. However, the present invention shall not to be limited in scope by the specific examples described herein. Indeed, various modifications of the invention in addition to those described herein will become readily apparent to those skilled in the art from the foregoing description and the example below. All such modifications fall within the scope of the appended claims.

### Example 1: Methods and Materials

The following methods and materials were used by the inventors in the subsequent examples, unless indicated otherwise.

### HEK293 Cell Culture

HEK293 cells were cultured in Dulbecco's Modified Eagle Medium (DMEM, Gibco™ 41965-039) supplement with 10% Fetal Bovine Serum (FBS, Gibco™, 10270), 1% Sodium Pyruvate (Gibco™, 11360070), 1% MEM NEAA (GibcoT^{M}, 11140035) and 0.5% Penicillin-Streptomycin (P-S; Sigma, P0781) and Passagel5-25 were used for experiments.

### Luminescent Cytotoxicity Assay

To test the cytotoxicity of compounds according to the present invention, HEK293 cells (70-80% confluent) were transfected 24 hours after plating with both GRIN1 and GRIN2 Å or GRIN2B, respectively (1:1, 0.2 mg/cm²) with Lipofectamine 2000 according to manufacturer's instructions. The relative number of dead cells in the population at the indicated time points after transfection was measured with the CytoTox-Glo™ Cytotoxicity assay (Promega, G9290) according to manufacturer's instruction with minor modification. Briefly, 10% of total medium were mixed with 10 μL AAF-amino luciferin to reach a final volume at 200 μL with water, the dead cell relative luminescence units (DRLU) was measured by GloMax (Promega) in a 96 well white bottom polystyrene microplate (Corning Costar®, 3912). After all the measurements, lysis reagents have been added to the cells and 10% of lysate was used for the total cell relative luminescence units (TRLU) measurement. Cell death was calculated by the following equations:

$$Cell death ( \% ) = \frac{10 * DRLU}{10 * TRLU + DRLU} * 100$$

For drug testing, P4, P8, P9, P13 and P15 were added to the medium at indicated concentration 6 h after transfection. DRLU, TRLU and cell death were measured and calculated 48 h after transfection.

### Primary Neuronal Cultures

Primary mouse hippocampal and cortical neurons were prepared and maintained as known. Briefly, hippocampi or cerebral cortex from P0 C57Bl/6NCrl mice were dissociated and plated at a density of 1.2*105/cm² in Growth Medium (GM), consisting of Neurobasal A medium (Gibco™, 10888022), 2% serum free B27™ Supplement (Gibco™, 17504044), 1% rat serum (Biowest, S2150), 0.5 mM L-Glutamine (Sigma, G7513) and 0.5% P-S. Cytosine β-D-arabinofuranoside (AraC; Sigma, C1768; 2.8 μM) was added on DIV3 to prevent the proliferation of glial cells. From DIV8, half of the medium was replaced by GM without Rat serum every 48 h until being used for experiments. 24 h before experiments, GM was replaced with transfection medium (10 mM HEPES, pH 7.4, 114 mM NaCl, 26.1 mM NaHCO₃, 5.3 mM KCl, 1 mM MgCl₂, 2 mM CaCl₂, 30 mM glucose, 1 mM glycine, 0.5 mM C₃H₃NaO₃, and 0.001% phenol red and 10% of phosphate-free Eagle's minimum essential medium, supplemented with 7.5 g/ml insulin, 7.5 g/ml transferrin and 7.5 ng/ml sodium selenite (ITS Liquid Media Supplement, Sigma-Aldrich Cat #I3146)). Primary hippocampal neurons were used in live cell imaging, cell death experiments, while cortical neurons were used for mRNA and protein extraction analysis.

Recombinant Adeno-Associated Viruses (rAAVs) and Constructs

All viral particles were produced and purified as known in the art. All TRPM4 derived peptides (comprising SEQ ID NO:5, or any of SEQ ID NO:46 to SEQ ID NO:56) were cloned into rAAV backbone by PCR. shRNA against mouse TRPM4 were designed by BLOCK-iT™ RNAi Designer from Thermofisher to target: ggacatcgcccaaagtgaact (SEQ ID NO:44, shTRPM4-1) and gcatccagagagggttcattc (SEQ ID NO:45, shTRPM4-2). Scramble shRNA (shSCR) has been tested and proven to have no known targets in mice. GPI anchor (LENGGTSLSEKTVLLLVTPFLAAAWSLHP, e.g. used in SEQ ID NO:53) sequences were synthesized by Eurofins Genomics (Ebersberg, Germany). All primers were synthesized and all plasmids were confirmed by sequencing by Eurofins Genomics.

### Mitochondrial Imaging

Coverslips with primary cultured neurons (DIV15-DIV17) were used to examine mitochondrial membrane potential (Ψm) and mitochondrial calcium signalling, which was performed at room temperature in CO₂-independent culture medium (CICM) containing: 10 mM HEPES, 140 mM NaCl, 2.5 mM KCl, 1.0 mM MgCl₂, 2.0 mM CaCl₂, 1.0 mM Glycine, 35.6 mM Glucose and 0.5 mM Na-pyruvate. All images were obtained by a cooled-CCD camera (iXon 887, Andor) on an upright microscope (BX51WI, Olympus). Fluorescence excitation was provided by a xenon arc lamp in combination with an excitation filter wheel (MT-20, Olympus). Data were collected using Cell\hat{ }R software (Olympus), analysed using ImageJ and quantified using Igor Pro (WaveMetrics). Ψm was detected with the small molecule fluorescence indicator Rhodamine 123 (Rh123; Molecular Probe™, R302). Primary cultured neurons were loaded with 4.3 μM Rh123 in CICM at 37° C. for 30 min, then washed and left in CICM for another 30 min before recording. At the end of each experiment, the mitochondrial uncoupler FCCP (5 μM, Sigma-Aldrich, Cat #C2920) was applied to the cells to reach the maximal Rh123 fluorescence intensity. Rh123 was imaged with 470±20 nm excitation and 525±25 nm emission wavelengths using a 20× objective. Rh123 fluorescence intensity was measured in the nucleus to minimize contamination from cytosolic mitochondrial signal and Rh123 fluorescence intensity was normalized to the maximum FCCP signal for each region of interest.

Gabazine induced mitochondrial Ca²⁺ response in primary cultured neurons is recorded and analysed as described in a previous study (Qiu et al, Nat Commun. 2013; 4:2034, incorporated herewith by reference) using a FRET calcium indicator 4mtD3cpv that specifically located to mitochondria. Briefly, mitochondrial Ca²⁺ levels were detected with FRET-based and mitochondrial targeted Ca²⁺ indicator 4mtD3cpv. 4mtD3cpv were excited at 430±12 nm (CFP) and 500±10 nm (YFP), and emission of CFP (470±12 nm) and YFP (535±15 nm) were separated and filtered using a DualView beam splitter (AHF Analysentechnik and MAG Biosystems), and all fluorescence images were recorded through a 20× water-immersion objective at 1 Hz.

### Quantification and Statistics

All statistics work was performed by Prism (GraphPad). All plotted data represent Mean±s.d. Two-Way ANOVA analysis were used for statistical analyses unless otherwise indicated.

### Reagents

Following reagents were used in this study MK-801 maleate (BN338, Biotrend). DL-APV (BN0858, Biotrend), NMDA (BN0385, Biotrend).

### Example 2: Knockdown of TRPM4 Protects Neurons from NMDA Receptor-Mediated Toxicity

In order to investigate the role of TRPM4 in NMDA receptor mediated excitotoxicity, the inventors used RNA interference strategies to knock down TRPM4. Cultured primary mouse hippocampus neurons were infected on day 3 in vitro (DIV3) with recombinant adeno-associated viruses (rAAVs) providing for expression of a scramble control (shSCR), shTRPM4-1 (SEQ ID NO:44) or shTRPM4-2 (SEQ ID NO:45). On DIV15-16, neurons were challenged with N-methyl-D-aspartate (NMDA, 20 μM) for 10 min. After NMDA wash out, neurons were kept in the culture medium for another 24 h before analysis. Knockdown of TRPM4 by both shRNAs against TRPM4 significantly protected neurons from NMDA induced excitotoxicity. Evidently, TRPM4 is thus involved in the process of NMDA receptor mediated excitotoxicity.

### Example 3: The N-Terminal Domain of Mouse TRPM4 Contains a Sequence which is Neuroprotective if Expressed in HEK293 Cells

In a next step, the inventors tried to identify regions in the mouse TRPM4 protein potentially involved in NMDA receptor mediated excitotoxicity. For this purpose, polypeptide fragments of mouse TRPM4 protein were generated and their impact on NMDA induced excitotoxicity analysed. To do so, primary neuron cultures were infected with respective rAAVs on DIV3, challenged with NMDA (20 μM) for 10 min on DIV17 and cell death assessed 24 hours later.

The first series of fragmentation experiments (comprising SEQ ID NO:46, SEQ ID NO:47, SEQ ID NO:48 and SEQ ID NO:49, respectively) revealed, that the N-terminal domain of mouse TRPM4 (SEQ ID NO:47) contains a neuroprotective element, which can prevent NMDA receptor induced cell death, if expressed in hippocampal neurons. In a further series of fragmentation experiments of SEQ ID NO:47 conducted in analogous manner as set out above, the inventors narrowed down the amino acid motif conferring the neuroprotective effect. The polypeptides comprising the following fragments were tested: SEQ ID NO:50, SEQ ID NO:51, SEQ ID NO:52, SEQ ID NO:5. As a result, only the most C-terminal part of the N-terminus of mouse TRPM4, aa 433-489, is surprisingly neuroprotective (SEQ ID NO:5), if expressed in hippocampal neurons.

### Example 4: Addition of a Membrane Anchor Increases Neuroprotective Effect of the Peptide According to SEQ ID NO:5

The sequence of SEQ ID NO:5 in TRPM4 is located in vivo just beneath the plasma membrane. Therefore, the inventors reasoned that a near-membrane location of a polypeptide comprising SEQ ID NO:5 might increase the neuroprotective effect of said polypeptide. To test this hypothesis, the inventors created a fusion protein comprising SEQ ID NO:5 and a GPI anchor, SEQ ID NO:57. The sequence of the fusion is given in SEQ ID NO:53. Neurons infected with rAAV and expressing SEQ ID NO:47 (control) or SEQ ID NO:53 were exposed to NMDA (20 μM) for 10 min on DIV15-16, with cell death assessed after 24 h. As a result it was shown that a membrane-anchor like GPI can increase the ability to protect neurons from excitotoxicity.

### Example 5: a Variant of the Sequence of SEQ ID NO:5 Also Reduces NMDA Receptor-Mediated Cell Toxicity

In a next step, the inventors created a mutant of SEQ ID NO:5, in which two adjacent phenylalanine residues where substituted by tyrosine residues (SEQ ID NO: 54). Furthermore, the inventors also assessed whether a region corresponding to SEQ ID NO:5 in mouse TRPM5 would also provide a neuroprotective effect. TRPM5 is a protein related to but nonetheless distinct to TRPM4. The region in TRPM5 corresponding to SEQ ID NO:5, SEQ ID NO:55, shares only about 60% sequence identity with SEQ ID NO:5. Neurons were infected on DIV3 with rAAVs expressing SEQ ID NO: 5, SEQ ID NO: 54 or SEQ ID NO:55 and exposed to NMDA (20 μM) for 10 min on DIV17, with cell death assessed 24 h later. As a result it was shown that the conservative double mutation harbouring the two tyrosine residues was only slightly less effective in reducing NMDA receptor-mediated cell toxicity than SEQ ID NO:5, while the more distantly related TRPM5 sequence of SEQ ID NO: 55 did not reduce NMDA receptor-mediated cell toxicity.

### Example 6: Exposure of Neurons to a Fusion Protein Comprising SEQ ID NO:5 Fused to a Protein Transduction Domain Protects Against NMDA Receptor-Mediated Cell Toxicity

In a further experiment, the inventors tested the fusion of SEQ ID NO:5 and protein transduction domain TAT (SEQ ID NO:42). The resulting fusion protein is referenced herein in SEQ ID NO:56. Cultured neurons were incubated with DMSO (vehicle), 1 μg SEQ ID NO:56 or 10 g SEQ ID NO:56 for 1 h before exposed to NMDA (20 μM) for 10 min on DIV15-16, with cell death assessed 24 h later. As a result, the fusion protein according to SEQ ID NO:56 protected neurons from NMDA excitotoxicity.

### Example 7: Viral Vector Mediated Expression of SEQ ID NO:5 in the Mouse Cortex Protects Against Middle Cerebral Artery Occlusion (MCAO)-Induced Brain Damage

Given the robust protective effect of SEQ ID NO:5 in cultured neurons, the inventors next analysed its neuroprotective potential in vivo using the middle cerebral artery occlusion (MCAO) mouse stroke model. This acute neurodegenerative disease was chosen because NMDA receptor-induced excitotoxicity contributes significantly to brain injury after induction of ischemic conditions. rAAVs containing expression cassettes for SEQ ID NO:5 were stereotactically delivered to the mouse cortex three weeks prior to MCAO and brain damage was quantified 7 days post-injury. The infarct volume of mice expressing SEQ ID NO:5 in the cortex was significantly smaller than that of the control mice injected intracerebrally with PBS.

### Methods:

Stereotactic intracerebral injection to the cortex of mice: C57BL/6N male mice (8 weeks±5 days old) weighing 25±1 g were randomly grouped and anaesthetized with a mixture of Sedin©, Midazolam and Fentanyl®-Janssen and placed on a rodent stereotactic frame on a heat pad temperature controlled by a ATC1000 DC rectal thermometer (World Precision Instruments, Berlin). rAAV-SEQ ID NO:5) was infused into the left cortex (coordinates relative to Bregma: first site: AP 0.2 mm; ML 2.0; DV −2.0; second site: AP 0.2; ML 2.0; DV −1.8; third site: AP 0.2; ML 3.0; DV −4.0; forth site: AP 0.2; ML 3.0; DV −3.5.) using a Ultra Micro Pump III (World Precision Instruments, Berlin) to drive a 10 μl Nanofil syringe (World Precision Instruments, Berlin). A total volume of 2 μl containing 1-2×10⁹ genomic particles of rAAV was injected at a rate of 200 nl/min, after which the needle was left in place at each injection site for 2 minutes to prevent backflow before needle withdrawal. Control mice were injected with the same volume of PBS using the same method. After stereotactic injections, mice were allowed to recover from anaesthesia by subcutaneous application of a mixture with ATIPAZOLE, Flumazenil and Naloxon and were returned to their home cages when they were fully awaken. Three weeks after stereotactic delivery of rAAVs animals were subjected to middle cerebral artery occlusion (MCAO).

MCAO: Middle cerebral artery occlusion (MCAO) induced a permanent distal occlusion of the middle cerebral artery (MCA). C57BL/6N male mice (8 weeks±5 days old) were anesthetized by intraperitoneal injection of 500 μl Tribromethanol (250 mg/kg bodyweight) and placed in a recumbent position. The animals were allowed to breathe spontaneously and were not ventilated. An incision was made from the left eye to the ear. When the temporal muscle was removed by electrocoagulation, the left MCA was visible through the semitranslucent temporal surface of the skull. After a small burr hole was made in the temporal bone with dental drill, the inner layer of the skull was removed with fine forceps, and the dura mater was opened carefully to expose the MCA. Care was taken to avoid damage to the brain tissue. NaCl solution (0.9%) was present in the area surrounding the MCA. A microbipolar electrocoagulator ERBE ICC 200 (Erbe Elektromedizin GmbH, Tubingen) was used to permanently occlude the MCA. During surgical procedures, rectal temperature was maintained at 37±0.5° C. with an ATC1000 DC temperature-controlled heat plate (World Precision Instruments, Berlin). After the incision was closed, mice were allowed to recover from anesthesia and returned to their home cages where the temperature was maintained at 37° C. by placing the cage on a HT 50 S heat plate (Minitub, Tiefenbach). In these conditions, animals were maintained homeothermic until fully recovery from anaesthesia. Sham-operated mice were subjected to identical procedures without the MCA occlusion. On day 7 after MCAO, animals were sacrificed under deep anaesthesia with Narcoren® and perfused intracardially with 20 ml NaCl solution (0.9%). The brains were removed from the skull and were immediately frozen on dry ice. Six consecutive 20 μm thick coronal cryo-sections were cut every 400 μm and subjected determination of total infarct volume using a standard silver staining technique. The silver stained sections were scanned at 1200 dpi and infarct area was measured by using ImageJ software (NIH Image). Surgery was performed and ischemic damage was measured by an investigator who had no knowledge of the treatment group, rAAV or recombinant protein was applied by stereotactic injection or intranasal delivery.

As a result, expression of a polypeptide comprising the sequence of SEQ ID NO:5 effectively reduced the infarct volumes, thereby protecting against middle cerebral artery occlusion (MCAO)-induced brain damage.

### Example 8: The Peptide Comprising the Sequence of SEQ ID NO:5 and a Variant Thereof Protect Against NMDA Receptor Induced Mitochondrial Membrane Potential Break Down

Mitochondrial dysfunction is a hallmark of NMDA receptor excitotoxicity and an early event en-route to neuronal death. A parameter that is often used to assess mitochondrial integrity is the mitochondrial membrane potential. A breakdown of the mitochondrial membrane potential can be observed after exposure of hippocampal or cortical neurons to NMDA and indicates excitotoxicity-associated mitochondrial dysfunction. Therefore, the inventors investigated the effect of polypeptides comprising the sequence of SEQ ID NO:5 or SEQ ID NO: 54 as well as of the control TRMP5 sequence (SEQ ID NO: 55) on mitochondrial membrane potential break down in primary mouse hippocampal neurons. Excitotoxicity leading to mitochondrial membrane potential break down was induced with bath application of 20 μM NMDA. After 11 minutes, the mitochondrial uncoupler, carbonylcyanide-p-trifluoromethoxyphenylhydrazone (FCCP) was added. Addition of FCCP leads to break-down of the mitochondrial membrane potential and served as a control of the test system.

As a result, polypeptides comprising the sequence of SEQ ID NO: 5 and SEQ ID NO: 54, respectively, were both capable of preventing NMDA receptor induced mitochondrial membrane potential breakdown, while the more distantly related TRPM5 sequence of SEQ ID NO: 55 was not capable of preventing NMDA receptor induced mitochondrial membrane potential breakdown.

### Example 9: The Peptides According to SEQ ID NO:5 and SEQ ID NO: 54 do not Affect Synaptic NMDA Receptor Signaling

In a further experiment the inventors assessed the impact of the polypeptide comprising the sequence of SEQ ID NO:5 and SEQ ID NO: 54, respectively, on Gabazine induced calcium influx (mitochondria), a measure of synaptic NMDA receptor signalling. The experiment was carried out in primary cultured neurons as described above. As a result, neither the polypeptide comprising the sequence of SEQ ID NO:5 nor the polypeptide comprising the sequence of SEQ ID NO: 54 did interfere with Gabazine induced calcium influx into mitochondria, indicating that neither of these polypeptides affects synaptic NMDA receptor signalling.

### Example 10: Virtual Screening for Compounds Potentially Binding to SEQ ID NO:5 in Mouse TRPM4

In a next step, the inventors tried to identify small molecule compounds capable of interacting with the above identified important domain of TRPM4 with the aim to identify compounds potentially capable of abrogating NMDA receptor-induced toxicity.

### Protein Structure

The protein structure used for this work was the 2.88 Å cryo-electron microscopy structure of mouse TRPM4 deposited in the Protein Data Bank (PDB ID: 6BCO). An alternative would be a human structure, such as 5WP6, 6BQR, 6BQV etc.). Prior to other activities, the structure was subjected to the Maestro Protein Preparation Wizard (Schrödinger Release 2017-3: Maestro, Schrödinger, LLC, New York, NY, 2017) to remove potential artifacts, add hydrogen atoms, and assign residue protonation states according to a pH of 7.0. Following preparation, all atoms not belonging to the protein (e.g. ATP molecules) were removed.

### Binding Site Definition

The region used for molecular docking was defined in 4 Å proximity to TRPM4 residues as considered relevant for protein activity (6BCO residues 633-650, 654, 655, 657, 664-668). See also amino acid residues 1 to 36 of SEQ ID NO:5.

### Molecular Docking

Docking to the protein structure was performed with Schrödinger Glide (Schrödinger Release 2017-3: Glide, Schrödinger, LLC, New York, NY, 2017). Initial compounds were docked in high-throughput virtual screening (HTVS) mode. Hits from HTVS (docking score of −5 kcal/mol or better required) were passed on to the more accurate and computationally expensive SP docking mode. SP hits with docking scores of −6 kcal/mol or better were subjected to a ligand strain calculation (energy difference between the pose and a minimum-energy solution conformation) with the OPLS3 force-field. Strains below 7 kcal/mol were generally desired with the exception of molecules with a large amount of rotatable bonds. Final compound selection was done on basis of docking scores, strain values, and visual inspection of the docked pose. Corresponding images were generated with the PyMOL Molecular Graphics System, Version 2.0 Schrödinger, LLC.

### Results

The screen yielded the following promising compounds:

TABLE 2
Promising candidate compounds obtained from docking

| | | | Internal |
|---|---|---|---|
| | | Docking score | strain |
| # | Compound | [kcal/mol] | [kcal/mol] |
| P4 | 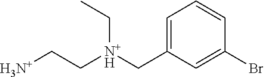 | −7.05 | 0.03 |
| P8 | 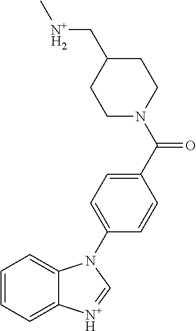 | −6.78 | 3.52 |
| P9 | 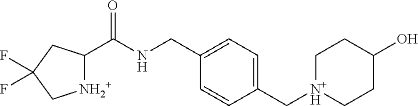 | −6.76 | 5.53 |
| P13 | 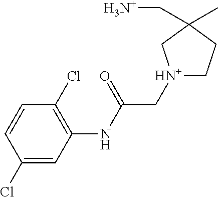 [SMILES: CC1(C[NH3+])CC[NH+](CC(=O)Nc2cc(Cl)ccc2Cl)C1] | −6.66 | 3.05 |
| P15 | 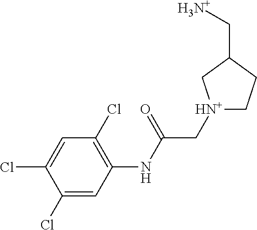 [SMILES: [NH3+]CC1CC[NH+](CC(=O)Nc2cc(Cl)c(Cl)cc2Cl)C1] | −6.62 | 4.46 |

### Example 11: Small Molecules According to the Present Invention Protect Against NMDA Receptor Induced Cell Toxicity

In this experiment the following compounds where tested for their suitability to protect HEK293 cells against NMDA receptor induced cytotoxicity:

| # | Compound |
|---|---|
| P4 | 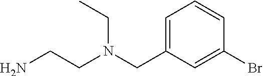 |
| P8 | 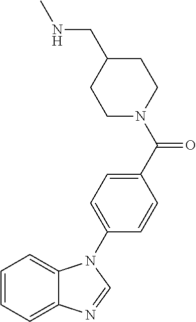 [SMILES: CNCC1CCN(C(=O)c2ccc(-n3cnc4ccccc43)cc2)CC1] |
| P9 | 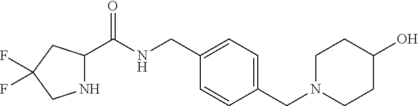 [SMILES: O=C(NCc1ccc(CN2CCC(O)CC2)cc1)C1CC(F)(F)CN1] |
| P13 | 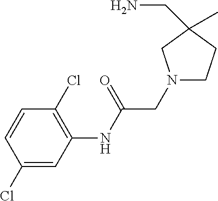 [SMILES: CC1(CN)CCN(CC(=O)Nc2cc(Cl)ccc2Cl)C1] |
| P15 | 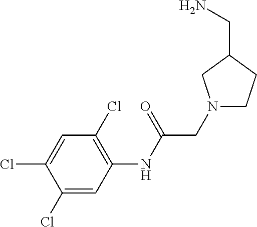 [SMILES: NCC1CCN(CC(=O)Nc2cc(Cl)c(Cl)cc2Cl)C1] |
| | 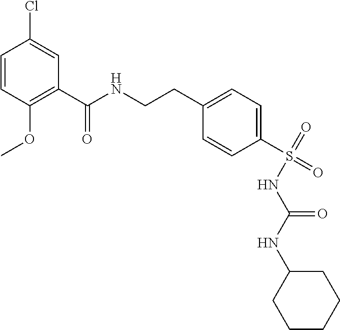 [SMILES: COc1ccc(Cl)cc1C(=O)NCCc1ccc(S(=O)(=O)NC(=O)NC2CCCCC2)cc1] |
| | (Glibenclamide) |

The experiment was carried out as described above. As a result, compounds P4, P8, P9, P13, and P15 reduced the level of NMDA receptor induced cell death. Glibenclamide is a known blocker of TRPM4 function and served as a positive control. The effect observed with these substances confirms the utility of virtual screening for identifying suitable candidate compounds for inhibiting NMDA receptor-mediated cell toxicity and the importance of the inventive TRPM4 motif for NMDA receptor-mediated cell toxicity.

### Example 12: Further Small Molecules According to the Present Invention

In view of the results obtained for compound P4 of example 11 above, the following additional nine variants thereof have been tested as essentially described above:

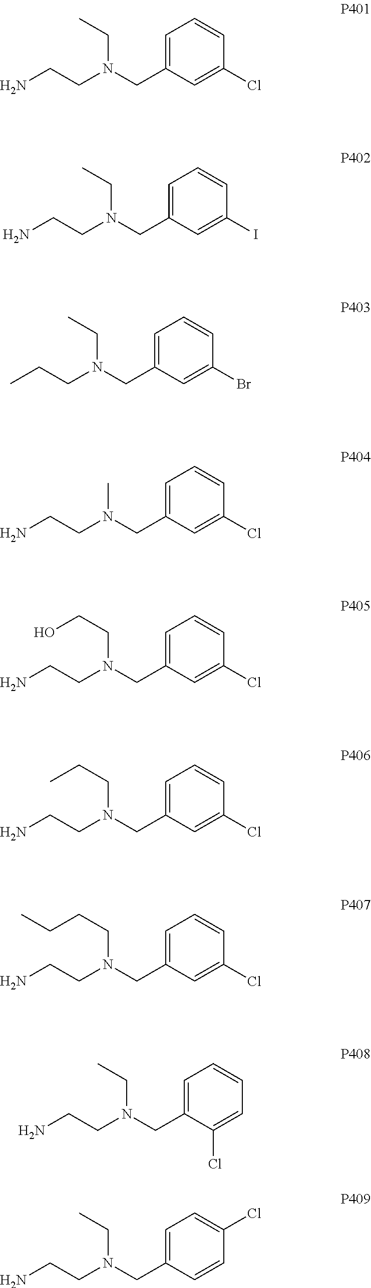

The experiment was carried out as described above. Briefly, neurons were pre-treated for 30 min with 10 μM of the indicated compound and then challenged with NMDA (20 μM) for 10 min (transient NMDA toxicity) or with NMDA (20 μM) for 24 hours (chronic NMDA toxicity). Cell death was assessed 24 hours after the NMDA challenge. For assessment of cell death, neurons were fixed with 4% paraformaldehyde, 4% sucrose in phosphate buffered saline (PBS) for 15 min, washed with PBS, and counterstained with Hoechst 33258 (1 μg/ml) for 10 min. The cells were mounted in Mowiol 4-88 and examined by fluorescence microscopy. The dead neurons were identified by amorphous or shrunken nuclei. As a result, the variants of compound P4, i.e. compounds P401 to P409, reduced the level of NMDA receptor induced cell death.

### Example 13: NMDA Receptor-Mediated Toxicity in TRPM4 Knock-Out HEK293 Cells

In a further experiment, the impact of a TRPM4 knock-out on NMDA receptor-mediated toxicity in HEK293 cells is was assessed. Briefly, HEK293 cells (both wild type line and TRPM4 knock-out line) (Ozhathil et al., British Journal of Pharmacology 175, 2504-2519) were cultured in Dulbecco's Modified Eagle Medium (DMEM, Gibco™, 41965-039) supplement with 10% Fetal Bovine Serum (FBS, GibcoT™, 10270), 1% Sodium Pyruvate (Gibco™, 11360070), 1% MEM NEAA (Gibco™, 11140035) and 0.5% Penicillin-Streptomycin (P-S; Sigma, P0781) and Passage 15-25 were used for experiments. To test the cytotoxicity of compounds according to the present invention, HEK293 cells (70-80% confluent) were transfected 24 hours after plating with both GRIN1 and GRIN2 Å or GRIN2B, respectively (1:1, 0.2 mg/cm2) with Lipofectamine 2000 according to manufacturer's instructions. The relative number of dead cells in the population at the indicated time points after transfection was measured with the CytoTox-Glo™ Cytotoxicity assay (Promega, G9290) according to manufacturer's instruction with minor modification. Briefly, 10% of total medium were mixed with 10 μL AAF-amino luciferin to reach a final volume at 200 μL with water, the dead cell relative luminescence units (DRLU) was measured by GloMax (Promega) in a 96 well white bottom polystyrene microplate (Corning Costar®, 3912). After all the measurements, lysis reagents have been added to the cells and 10% of lysate was used for the total cell relative luminescence units (TRLU) measurement. Cell death was calculated by the following equations:

$$Cell death ( \% ) = \frac{10 * DRLU}{10 * TRLU + DRLU} * 100$$

As a result NMDA receptor-mediated toxicity was much reduced in the TRPM4 knock-out HEK293 cells as compared to wild type HEK293 cells. This aligns with the results reported in Example 2.

### Example 14: Impact of Compound P4 and P15 on NMDA-Induced Calcium Transients

In a further experiment, the impact of P4 and P15 on NMDA-induced calcium transients was assessed. Briefly, for calcium imaging, primary hippocampal neurons on coverslips were loaded with the cell-permeable, high-affinity ratiometric calcium indicator Fura2-AM (Invitrogen™ F1221), at 1 μM in CO₂-independent culture medium (CICM; CICM contains: 10 mM HEPES, 140 mM NaCl, 2.5 mM KCl, 1.0 mM MgCl₂, 2.0 mM CaCl₂, 1.0 mM Glycine, 35.6 mM Glucose and 0.5 mM Na-pyruvate) for 30 min at 37° C., then washed and left in CICM for another 30 min to allow for de-esterification. Fura2 was excited at 340/11 nm and 380/11 nm, and fluorescence emission was obtained from a 40× UV compatible objective (LUMPLFLN, Olympus) through a 510/20 nm emission filter. For quantification, ImageJ was used to calculate average background-subtracted fluorescence intensities for 340 and 380 nm excitation (F₃₄₀ and F₃₈₀) from each neuron. Intracellular calcium levels were plotted as F₃₄₀/F₃₈₀ ratios over time, from which the NMDA response amplitude and area under the curve (AUC) was calculated to quantify NMDA-induced calcium influx.

Strikingly, unlike the classical NMDA receptor blocker, MK-801, which completely blocked NMDA-induced calcium transients, neither compound P4 nor compound P15 reduced NMDA-induced calcium transients in hippocampal neurons. Thus, P4 and P15 block NMDA receptor excitotoxicity, but without compromising NMDA receptor calcium channel function, which is essential for the physiological role of NMDA receptors in synapse-to-nucleus signaling, gene regulation, and cognitive functions, including learning and memory.

### Example 15: Impact of Compound P4 on NMDA Receptor/TRPM4 Complex Immunoprecipitation

In a further experiment it was assessed whether compound P4 has any impact on NMDA receptor/TRPM4 complex formation. For this purpose, co-immunoprecipitation experiments using brain lysates from the mouse cortex where carried out. Briefly, cortical lysates were obtained from control mice and from mice 2 h, 6 h and 24 h following intraperitoneal injection of compound P4 (40 mg/kg) in immunoprecipitation buffer (10 mM Tris, pH 8.0, 150 mM NaCl, 1 mM EDTA, 1% NP-40, 10% glycerol with Protease Inhibitor Cocktail, Roche) for 60 min. The lysate was then centrifuged for 12 min at 1200 g to remove cell debris and nuclei. The mixture of supernatant was incubated with anti-TRPM4 antibodies overnight. Pierce™ Protein A Magnetic Beads were added to the mixture and mixed another 12 h, followed by 3 washes with immunoprecipitation buffer. The precipitates were subsequently boiled in protein loading buffer and separated in 7.5% SDS-PAGE.

The inventors found a reduction in NMDA receptor/TRPM4 complex formation of 51% at 2 h and 61% at 6 h after a single intraperitoneal (i.p.) injection of 40 mg/kg of compound P4. 24 h after i.p. injection of compound P4 the NMDA receptor/TRPM4 complex had reformed.

### Example 16: Impact of Compound P4 on NMDA-Induced Degeneration of Retinal Ganglion Cells (RGCs)

The inventors also assessed, whether compound P4 can protect retinal ganglion cells against NMDA-induced degeneration. For this purpose, 28 C57BL/6J mice (25±3.5 g) were randomly allocated to two groups. All mice received vehicle (sunflower oil containing 5% ethanol) or P4 (40 mg/kg, dissolved in sunflower oil containing 5% ethanol) through intraperitoneal injection at −16h, −3h, 0 h, +3 h and +24 h in a volume of 50 μL each injection. At 0 h, mice received 20 nmol of NMDA (total volume 2.0 μL) by intravitreal injection in the left eye and saline (total volume 2.0 μL) in the right eye. Both eyes were removed from euthanized mice 7 days after intravitreal injections and fixed in formalin for 15 min before retinas were dissected and processed for whole mount immunostaining. Retinas were incubated in blocking solution (10% FBS, 1% Triton-X 100 in PBS) for 6 h, followed by 24 h incubation with anti-Brn3a antibody in blocking solution at 4° C. Retinas were washed 3 times with PBS and incubated with Donkey anti-rabbit Alexa Fluor-594 for 24 h at room temperature. Retinas were washed again, cut and mounted onto slides. For each retina, images were obtained from eight fields (554 μm×554 μm) around the peripheral retina (two from each quadrant and located at ~600 μm or ~1400 μm to macular hole) to minimize the location-associated variability in RGCs density. All images were obtained using Las X software via an HC PL APO 20× objective on a Leica TCS SP8LIA in a DM6 CFS upright confocal microscope. Brn3a-positive cells were identified and counted with a macro in Cellprofiler. The data analysis was performed on a single-blind basis without knowledge of treatment.

The inventors found that compound P4 reduced retinal ganglion cell (RGC) degeneration after intravitreal injection of mice with NMDA (20 nmol).

### Example 17: Impact of Compound P4 and Compound P15 on TRPM4 Channel Function

To assess any direct impact of compound P4 and compound P15 on TRPM4 channel function independent of NMDA receptors, the inventors used the prostate cancer cell line PC3 and patch clamp recordings. PC3 cells are known to express TRPM4 channels (C. Holzmann et al., Oncotarget. 6, 41783-93 (2015)). TRPM4 currents in turn are characterized by their calcium dependence and outward rectification (P. Launay et al., Cell. 109, 397-407 (2002)). Briefly, whole-cell patch clamp recordings were made from PC3 cells plated on 12 mm round coverslips secured with a platinum ring in a recording chamber (OAC-1, Science Products GmbH) mounted on a fixed-stage upright microscope (BX51WI, Olympus). Coverslips were submerged with continuously flowing (3 ml/min) 32-35° C. extracellular solution (in mM: NaCl, 156; MgCl₂, 2; CaCl₂), 1.5; HEPES, 10; glucose, 10). Patch electrodes (3-4 MΩ) were made from 1.5 mm borosilicate glass and filled with cesium-based solutions (in mM: CsCl, 145; NaCl, 8; HEPES, 10; MgCl₂, 1; plus either EGTA, 0.2 for a free Ca²⁺ concentration of zero; or EGTA, 10 and CaCl₂), 9.4 for a calculated free Ca²⁺ concentration of 10 μM; Maxchelator, Stanford University). Recordings were made with a Multiclamp 700B amplifier, digitized through a Digidata 1550B and acquired and analyzed using pClamp 10 software (Molecular Devices). Access resistance (range: 10-20 MΩ) was monitored regularly during voltage clamp recordings and data was rejected if changes greater than 20% occurred.

As a result, 10 μM Ca²⁺ activated a TRPM4-like outwardly rectifying current in PC3 cells and this current was not affected by P4 or P15. Thus, neither P4 nor P15 compromises TRPM4 channel function per se.

1. A method for inhibiting NMDA receptor mediated toxicity in a subject in need thereof, wherein the method comprises administering an effective amount of a compound that is an inhibitor of NMDA receptor/TRPM4 complex formation to the subject.

2. The method according to claim 1, wherein the subject suffers from a neurological disease.

3. The method according to claim 2, wherein the neurological disease is selected from the group consisting of stroke, Alzheimer's disease (AD), amyotrophic lateral sclerosis (ALS), Huntington's disease (HD), traumatic brain injury, multiple sclerosis, glutamate induced excitotoxicity, dystonia, epilepsy, optic nerve disease, diabetic retinopathy, glaucoma, neuropathic pain, anti-NMDA receptor encephalitis, viral encephalopathy, vascular dementia, microangiopathy, Binswanger's disease, cerebral ischemia, hypoxia, Parkinson's disease, schizophrenia, depression, cerebral malaria, toxoplasmosis-associated brain damage, HIV infection-associated brain damage, Zika virus infection-associated brain damage and a brain tumour.

4. The method according to claim 1, wherein the compound is a small molecule, a nucleic acid or a polypeptide.

5. The method according to claim 4, wherein the compound is a small molecule according to the following formula: 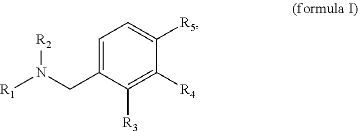
wherein:
R₁ and R₂ are each independently selected from hydrogen, alkyl_{(C≤12)}, and substituted alkyl_{(C≤12)}; and
R₃, R₄ and R₅ are each independently selected from hydrogen, hydroxy and halo; or
a pharmaceutically acceptable salt, solvate, polymorph, tautomer, racemate, or enantiomer thereof.

6. The method according to claim 4, wherein the compound is a small molecule selected from the group consisting of: 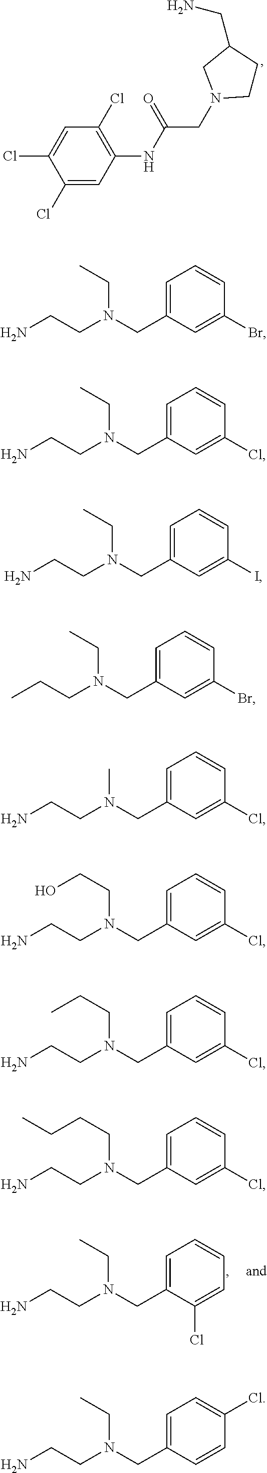
and a pharmaceutically acceptable salt, solvate, polymorph, tautomer, racemate, or enantiomer of any of these compounds.

7. The method according to claim 1, wherein the subject is a human being.

8. A method for inhibiting NMDA receptor/TRPM4 complex formation, the method comprising administering an effective amount of a compound that is an inhibitor of NMDA receptor/TRPM4 complex formation to a subject in need thereof.

9. The method according to claim 8, wherein the subject suffers from a neurological disease.

10. The method according to claim 9, wherein the neurological disease involves NMDA receptor mediated toxicity.

11. The method according to claim 9, wherein the neurological disease is selected from the group consisting of stroke, Alzheimer's disease (AD), amyotrophic lateral sclerosis (ALS), Huntington's disease (HD), traumatic brain injury, multiple sclerosis, glutamate induced excitotoxicity, dystonia, epilepsy, optic nerve disease, diabetic retinopathy, glaucoma, neuropathic pain, anti-NMDA receptor encephalitis, viral encephalopathy, vascular dementia, microangiopathy, Binswanger's disease, cerebral ischemia, hypoxia, Parkinson's disease, schizophrenia, depression, cerebral malaria, toxoplasmosis-associated brain damage, HIV infection-associated brain damage, Zika virus infection-associated brain damage and a brain tumour.

12. The method according to claim 1, wherein the compound is a small molecule, a nucleic acid or a polypeptide.

13. The method according to claim 8, wherein the compound is a small molecule according to the following formula: 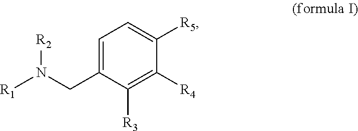
wherein:
R₁ and R₂ are each independently selected from hydrogen, alkyl_{(C≤12)}, and substituted alkyl_{(C≤12)}; and
R₃, R₄ and R₅ are each independently selected from hydrogen, hydroxy and halo; or
a pharmaceutically acceptable salt, solvate, polymorph, tautomer, racemate, or enantiomer thereof.

14. The method according to claim 8, wherein the compound is a small molecule selected from the group consisting of: 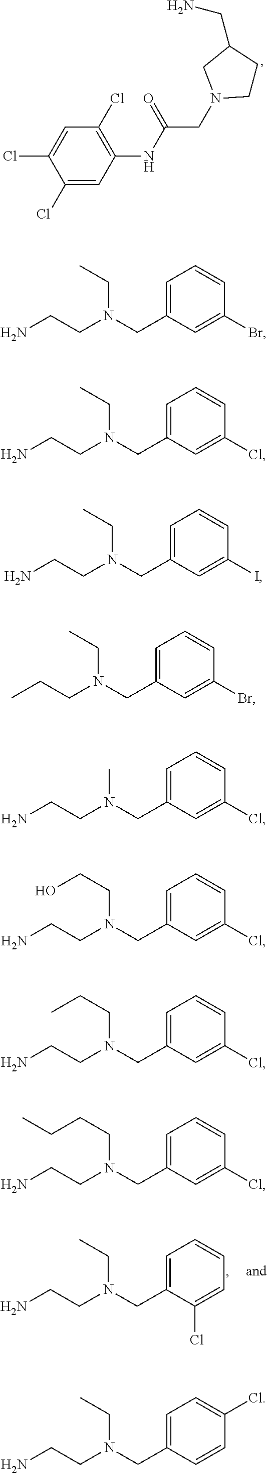
and a pharmaceutically acceptable salt, solvate, polymorph, tautomer, racemate, or enantiomer of any of these compounds.

15. The method according to claim 8, wherein the subject is a human being.


SEQUENCE LISTING (58 sequences)

SEQ ID NO: 1 - protein, 1214 aa, Homo sapiens
MVVPEKEQSWIPKIFKKKTCTTFIVDSTDPGGTLCQCGRPRTAHPAVAMEDAFGAAVVTVWDSDAHTTEKPTDAYGELDFTGAGRKHSNFLRLSDRTDPAAVYSLVTRTWGFRAPNLVVSVLGGSGGPVLQTWLQDLLRRGLVRAAQSTGAWIVTGGLHTGIGRHVGVAVRDHQMASTGGTKVVAMGVAPWGVVRNRDTLINPKGSFPARYRWRGDPEDGVQFPLDYNYSAFFLVDDGTHGCLGGENRFRLRLESYISQQKTGVGGTGIDIPVLLLLIDGDEKMLTRIENATQAQLPCLLVAGSGGAADCLAETLEDTLAPGSGGARQGEARDRIRRFFPKGDLEVLQAQVERIMTRKELLTVYSSEDGSEEFETIVLKALVKACGSSEASAYLDELRLAVAWNRVDIAQSELFRGDIQWRSFHLEASLMDALLNDRPEFVRLLISHGLSLGHFLTPMRLAQLYSAAPSNSLIRNLLDQASHSAGTKAPALKGGAAELRPPDVGHVLRMLLGKMCAPRYPSGGAWDPHPGQGFGESMYLLSDKATSPLSLDAGLGQAPWSDLLLWALLLNRAQMAMYFWEMGSNAVSSALGACLLLRVMARLEPDAEEAARRKDLAFKFEGMGVDLFGECYRSSEVRAARLLLRRCPLWGDATCLQLAMQADARAFFAQDGVQSLLTQKWWGDMASTTPIWALVLAFFCPPLIYTRLITFRKSEEEPTREELEFDMDSVINGEGPVGTADPAEKTPLGVPRQSGRPGCCGGRCGGRRCLRRWFHFWGAPVTIFMGNVVSYLLFLLLFSRVLLVDFQPAPPGSLELLLYFWAFTLLCEELRQGLSGGGGSLASGGPGPGHASLSQRLRLYLADSWNQCDLVALTCFLLGVGCRLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQRYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 2 - protein, 1069 aa, Homo sapiens
MVVPEKEQSWIPKIFKKKTCTTFIVDSTDPGGTLCQCGRPRTAHPAVAMEDAFGAAVVTVWDSDAHTTEKPTDAYGELDFTGAGRKHSNFLRLSDRTDPAAVYSLVTRTWGFRAPNLVVSVLGGSGGPVLQTWLQDLLRRGLVRAAQSTGAWIVTGGLHTGIGRHVGVAVRDHQMASTGGTKVVAMGVAPWGVVRNRDTLINPKGSFPARYRWRGDPEDGVQFPLDYNYSAFFLVDDGTHGCLGGENRFRLRLESYISQQKTGVGGTGIDIPVLLLLIDGDEKMLTRIENATQAQLPCLLVAGSGGAADCLAETLEDTLAPGSGGARQGEARDRIRRFFPKGDLEVLQAQVERIMTRKELLTVYSSEDGSEEFETIVLKALVKACGSSEASAYLDELRLAVAWNRVDIAQSELFRGDIQWRSFHLEASLMDALLNDRPEFVRLLISHGLSLGHFLTPMRLAQLYSAAPSNSLIRNLLDQASHSAGTKAPALKGGAAELRPPDVGHVLRMLLGKMCAPRYPSGGAWDPHPGQGFGESMYLLSDKATSPLSLDAGLGQAPWSDLLLWALLLNRAQMAMYFWEMGSNAVSSALGACLLLRVMARLEPDAEEAARRKDLAFKFEGMGVDLFGECYRSSEVRAARLLLRRCPLWGDATCLQLAMQADARAFFAQDGVQSLLTQKWWGDMASTTPIWALVLAFFCPPLIYTRLITFRKSEEEPTREELEFDMDSVINGEGPVGLTPGLYHLGRTVLCIDFMVFTVRLLHIFTVNKQLGPKIVIVSKMMKDVFFFLFFLGVWLVAYGVATEGLLRPRDSDFPSILRRVFYRPYLQIFGQIPQEDMDVALMEHSNCSSEPGFWAHPPGAQAGTCVSQYANWLVVLLLVIFLLVANILLVNLLIAMFSYTFGKVQGNSDLYWKAQRYRLIREFHSRPALAPPFIVISHLRLLLRQLCRRPRSPQPSSPALEHFRVYLSKEAERKLLTWESVHKENFLLARARDKRESDSERLKRTSQKVDLALKQLGHIREYEQRLKVLEREVQQCSRVLGWVAEALSRSALLPPGGPPPPDLPGSKD

SEQ ID NO: 3 - protein, 57 aa, Homo sapiens
SSEVRAARLLLRRCPLWGDATCLQLAMQADARAFFAQDGVQSLLTQKWWGDMASTTP

SEQ ID NO: 4 - protein, 57 aa, synthetic construct
  REGION 1..57: note = Consensus sequence on basis of SEQ ID NOs 3 and 5 to 41
  VAR_SEQ 1: note = X can be Asn or Ser
  VAR_SEQ 2: note = X can be Asn or Ser
  VAR_SEQ 4: note = X can be Asp, Glu, Tyr, Lys, His, Thr, Val or Asn
  VAR_SEQ 5: note = X can be Arg or Trp
  VAR_SEQ 6: note = X can be Ala or Ser
  VAR_SEQ 7: note = X can be Ala, Ser or Asn
  VAR_SEQ 8: note = X can be Arg, Gly or His
  VAR_SEQ 11: note = X can be Leu, Val or Ile
  VAR_SEQ 12: note = X can be Arg or Trp
  VAR_SEQ 16: note = X can be Leu or Phe
  VAR_SEQ 19: note = X can be Asp, Glu or Gly
  VAR_SEQ 20: note = X can be Ala or Ser
  VAR_SEQ 23: note = X can be Leu or Phe
  VAR_SEQ 24: note = X can be Gln or His
  VAR_SEQ 27: note = X can be Met or Thr
  VAR_SEQ 31: note = X can be Ala or Ser
  VAR_SEQ 33: note = X can be Ala or Ser
  VAR_SEQ 35: note = X can be Leu or Phe
  VAR_SEQ 40: note = X can be Val or Ile
  VAR_SEQ 42: note = X can be Ser or Phe
  VAR_SEQ 43: note = X can be Leu or Val
  VAR_SEQ 51: note = X can be Asp, Glu or His
  VAR_SEQ 53: note = X can be Ala or Asp
  VAR_SEQ 55: note = X can be Thr or Ser
  VAR_SEQ 56: note = X can be Thr or Asn
XXEXXXXXLLXXRCPXWGXXTCXXLAXQADXRXFXAQDGXQXXLTQKWWGXMXSXXP

SEQ ID NO: 5 - protein, 57 aa, Mus musculus
NSEERAARLLLRRCPLWGEATCLQLAMQADARAFFAQDGVQSLLTQKWWGEMDSTTP

SEQ ID NO: 6 - protein, 57 aa, Peromyscus maniculatus; identical to SEQ ID NO: 5

SEQ ID NO: 7 - protein, 57 aa, Mus caroli
NSEDRAARLLLRRCPLWGEATCLQLAMQADARAFFAQDGVQSLLTQKWWGEMDSTTP

SEQ ID NO: 8 - protein, 57 aa, Cricetulus griseus
NSEERAARLLLRRCPLWGEATCLQLAMQADARSFFAQDGVQSLLTQKWWGDMDSTTP

SEQ ID NO: 9 - protein, 57 aa, Jaculus jaculus
NNEDRAARLLLRRCPLWGEATCLQLAMQADARAFFAQDGVQSLLTQKWWGEMDSTTP

SEQ ID NO: 10 - protein, 57 aa, Canis lupus
  source 1..57: note = familiaris
SSEERAARLLLRRCPLWGDATCLQLAMQADARAFFAQDGVQSLLTQKWWGEMDSTTP

SEQ ID NO: 11 - protein, 57 aa, Hipposideros armiger
NSEDRAARLLLRRCPFWGDATCLQLAMQADARAFFAQDGVQSLLTQKWWGEMDSTTP

SEQ ID NO: 12 - protein, 57 aa, Rattus norvegicus
NSEYRAARLLLRRCPLWGEATCLQLAMQADARAFFAQDGVQSLLTQKWWGEMDSTNP

SEQ ID NO: 13 - protein, 57 aa, Acinonyx jubatus
SSEKRAARLLLRRCPLWGDATCLQLAMQADARAFFAQDGVQSLLTQKWWGEMDSTTP

SEQ ID NO: 14 - protein, 57 aa, Odobenus rosmarus
  source 1..57: note = divergens
SSEERAARLLVRRCPLWGDATCLQLAMQADARAFFAQDGVQSLLTQKWWGEMDSTTP

SEQ ID NO: 15 - protein, 57 aa, Neomonachus schauinslandi; identical to SEQ ID NO: 14

SEQ ID NO: 16 - protein, 57 aa, Manis javanica
SSEHRAARLLIRRCPLWGDATCLQLAMQADARAFFAQDGVQSLLTQKWWGEMDSTTP

SEQ ID NO: 17 - protein, 57 aa, Marmota marmota
  source 1..57: note = marmota
SSEDRAARLLLRRCPLWGDATCLQLAMQADARAFFAQDGVQSLLTQKWWGEMDSTTP

SEQ ID NO: 18 - protein, 57 aa, Enhydra lutris; identical to SEQ ID NO: 13
  source 1..57: note = kenyoni

SEQ ID NO: 19 - protein, 57 aa, Ictidomys tridecemlineatus; identical to SEQ ID NO: 17

SEQ ID NO: 20 - protein, 57 aa, Felis catus; identical to SEQ ID NO: 13

SEQ ID NO: 21 - protein, 57 aa, Heterocephalus glaber
SSEERASRLLLRRCPLWGDATCLQLAMQADARAFFAQDGVQSLLTQKWWGEMDSTTP

SEQ ID NO: 22 - protein, 57 aa, Capra hircus
SSEERSARLLLRRCPLWGDATCLQLATQADARAFFAQDGVQSLLTQKWWGEMDSTTP

SEQ ID NO: 23 - protein, 57 aa, Equus asinus
SSEERASRLLLRRCPLWGDATCFQLAMQADARAFFAQDGIQSLLTQKWWGEMDSTTP

SEQ ID NO: 24 - protein, 57 aa, Rhinolophus sinicus
NSEDRAARLLLRRCPFWGDATCFQLAMQADARAFFAQDGVQSLLTQKWWGEMDSSTP

SEQ ID NO: 25 - protein, 57 aa, Ovis aries; identical to SEQ ID NO: 22

SEQ ID NO: 26 - protein, 57 aa, Mesocricetus auratus
NSEERAAGLLLRRCPLWGEATCLQLAMQADARSFFAQDGVQFLLTQKWWGEMDSTTP

SEQ ID NO: 27 - protein, 57 aa, Bison bison; identical to SEQ ID NO: 22
  source 1..57: note = bison

SEQ ID NO: 28 - protein, 57 aa, Bos taurus; identical to SEQ ID NO: 22

SEQ ID NO: 29 - protein, 57 aa, Bubalus bubalis; identical to SEQ ID NO: 22

SEQ ID NO: 30 - protein, 57 aa, Erinaceus europaeus
SSEDRANRLLLRRCPLWGDATCLQLAMQADARAFFAQDGVQSLLTQKWWGEMDSTTP

SEQ ID NO: 31 - protein, 57 aa, Cavia porcellus
SNEHRASRLLLRRCPLWGDATCLQLAMQADSRAFFAQDGVQSLLTQKWWGEMDSTTP

SEQ ID NO: 32 - protein, 57 aa, Equus caballus
SSEERASRLLLRRCPLWGGATCFQLAMQADARAFFAQDGIQSLLTQKWWGEMDSTTP

SEQ ID NO: 33 - protein, 57 aa, Camelus dromedarius
SSEDRAARLLLRRCPLWGDSTCLQLATQADARAFFAQDGVQSLLTQKWWGEMDSTTP

SEQ ID NO: 34 - protein, 57 aa, Theropithecus gelada; identical to SEQ ID NO: 3

SEQ ID NO: 35 - protein, 57 aa, Orcinus orca
SSEERAAHLLLWRCPLWGDATCLHLAMQADARAFFAQDGVQSLLTQKWWGEMDSTTP

SEQ ID NO: 36 - protein, 57 aa, Chlorocebus sabaeus; identical to SEQ ID NO: 3

SEQ ID NO: 37 - protein, 57 aa, Chinchilla lanigera
SSETRASRLLLRRCPLWGDATCLQLAMQADARAFLAQDGVQSVLTQKWWGEMDSTTP

SEQ ID NO: 38 - protein, 57 aa, Elephantulus edwardii
SNEKWAARLLLRRCPLWGDATCLQLAMQADSRAFFAQDGVQSLLTQKWWGEMDSTTP

SEQ ID NO: 39 - protein, 57 aa, Gorilla gorilla
  source 1..57: note = gorilla
SNEVRAARLLLRRCPLWGDATCLQLAMQADARAFFAQDGVQSLLTQKWWGDMASTTP

SEQ ID NO: 40 - protein, 57 aa, Sus scrofa
NSENRAARLLLRRCPLWGDATCLQLATQADARAFFAQDGVQSLLTQKWWGHMDSTTP

SEQ ID NO: 41 - protein, 57 aa, Pan troglodytes; identical to SEQ ID NO: 3

SEQ ID NO: 42 - protein, 9 aa, Human immunodeficiency virus
RKKRRQRRR

SEQ ID NO: 43 - protein, 9 aa, unidentified
  REGION 1..9: note = HA tag
YPYDVPDYA

SEQ ID NO: 44 - RNA, 21 nt, Mus musculus
  source 1..21: mol_type = other RNA
ggacatcgcccaaagtgaact

SEQ ID NO: 45 - RNA, 21 nt, Mus musculus
  source 1..21: mol_type = other RNA
gcatccagagagggttcattc

SEQ ID NO: 46 - protein, 347 aa, Mus musculus
MVGPEKEQSWIPKIFRKKVCTTFIVDLSDDAGGTLCQCGQPRDAHPSVAVEDAFGAAVVTEWNSDEHTTEKPTDAYGDLDFTYSGRKHSNFLRLSDRTDPATVYSLVTRSWGFRAPNLVVSVLGGSGGPVLQTWLQDLLRRGLVRAAQSTGAWIVTGGLHTGIGRHVGVAVRDHQTASTGSSKVVAMGVAPWGVVRNRDMLINPKGSFPARYRWRGDPEDGVEFPLDYNYSAFFLVDDGTYGRLGGENRFRLRFESYVAQQKTGVGGTGIDIPVLLLLIDGDEKMLKRIEDATQAQLPCLLVAGSGGAADCLVETLEDTLAPGSGGLRRGEARDRIRRYFPKGDPEV

SEQ ID NO: 47 - protein, 342 aa, Mus musculus
LQAQVERIMTRKELLTVYSSEDGSEEFETIVLRALVKACGSSEASAYLDELRLAVAWNRVDIAQSELFRGDIQWRSFHLEASLMDALLNDRPEFVRLLISHGLSLGHFLTPVRLAQLYSAVSPNSLIRNLLDQASHASSSKSPPVNGTVELRPPNVGQVLRTLLGETCAPRYPARNTRDSYLGQDHRENDSLLMDWANKQPSTDASFEQAPWSDLLIWALLLNRAQMAIYFWEKGSNSVASALGACLLLRVMARLESEAEEAARRKDLAATFESMSVDLFGECYHNSEERAARLLLRRCPLWGEATCLQLAMQADARAFFAQDGVQSLLTQKWWGEMDSTTP

SEQ ID NO: 48 - protein, 347 aa, Mus musculus
IWALLLAFFCPPLIYTNLIVFRKSEEEPTQKDLDFDMDSSINGAGPPGTVEPSAKVALERRQRRRPGRALCCGKFSKRWSDFWGAPVTAFLGNVVSYLLFLLLFAHVLLVDFQPTKPSVSELLLYFWAFTLLCEELRQGLGGGWGSLASGGRGPDRAPLRHRLHLYLSDTWNQCDLLALTCFLLGVGCRLTPGLFDLGRTVLCLDFMIFTLRLLHIFTVNKQLGPKIVIVSKMMKDVFFFLFFLCVWLVAYGVATEGILRPQDRSLPSILRRVFYRPYLQIFGQIPQEEMDVALMIPGNCSMERGSWAHPEGPVAGSCVSQYANWLVVLLLIVFLLVANILLLNLLI

SEQ ID NO: 49 - protein, 177 aa, Mus musculus
AMFSYTFSKVHGNSDLYWKAQRYSLIREFHSRPALAPPLIIISHVRLLIKWLRRCRRCRRANLPASPVFEHFRVCLSKEAERKLLTWESVHKENFLLAQARDKRDSDSERLKRTSQKVDTALKQLGQIREYDRRLRGLEREVQHCSRVLTWMAEALSHSALLPPGAPPPPSPTGSKD

SEQ ID NO: 50 - protein, 122 aa, Mus musculus
LQAQVERIMTRKELLTVYSSEDGSEEFETIVLRALVKACGSSEASAYLDELRLAVAWNRVDIAQSELFRGDIQWRSFHLEASLMDALLNDRPEFVRLLISHGLSLGHFLTPVRLAQLYSAVS

SEQ ID NO: 51 - protein, 100 aa, Mus musculus
SLGHFLTPVRLAQLYSAVSPNSLIRNLLDQASHASSSKSPPVNGTVELRPPNVGQVLRTLLGETCAPRYPARNTRDSYLGQDHRENDSLLMDWANKQPST

SEQ ID NO: 52 - protein, 124 aa, Mus musculus
SYLGQDHRENDSLLMDWANKQPSTDASFEQAPWSDLLIWALLLNRAQMAIYFWEKGSNSVASALGACLLLRVMARLESEAEEAARRKDLAATFESMSVDLFGECYHNSEERAARLLLRRCPLWG

SEQ ID NO: 53 - protein, 371 aa, synthetic construct
  REGION 1..371: note = Fusion protein of SEQ ID NO: and SEQ ID NO:57
LQAQVERIMTRKELLTVYSSEDGSEEFETIVLRALVKACGSSEASAYLDELRLAVAWNRVDIAQSELFRGDIQWRSFHLEASLMDALLNDRPEFVRLLISHGLSLGHFLTPVRLAQLYSAVSPNSLIRNLLDQASHASSSKSPPVNGTVELRPPNVGQVLRTLLGETCAPRYPARNTRDSYLGQDHRENDSLLMDWANKQPSTDASFEQAPWSDLLIWALLLNRAQMAIYFWEKGSNSVASALGACLLLRVMARLESEAEEAARRKDLAATFESMSVDLFGECYHNSEERAARLLLRRCPLWGEATCLQLAMQADARAFFAQDGVQSLLTQKWWGEMDSTTPLENGGTSLSEKTVLLLVTPFLAAAWSLHP

SEQ ID NO: 54 - protein, 57 aa, synthetic construct
  REGION 1..57: note = Mutant of SEQ ID NO:5 with F34Y and F35Y
NSEERAARLLLRRCPLWGEATCLQLAMQADARAYYAQDGVQSLLTQKWWGEMDSTTP

SEQ ID NO: 55 - protein, 57 aa, Mus musculus
NSEDRAFALLVRRNHSWSRTTCLHLATEADAKAFFAHDGVQAFLTKIWWGDMATGTP

SEQ ID NO: 56 - protein, 69 aa, synthetic construct
  REGION 1..69: note = Fusion protein composed of TAT peptide and SEQ ID NO: 5
RKKRRQRRRSQPNSEERAARLLLRRCPLWGEATCLQLAMQADARAFFAQDGVQSLLTQKWWGEMDSTTP

SEQ ID NO: 57 - protein, 29 aa, unidentified
  REGION 1..29: note = Glycosylphosphatidylinositol (GPI) signal anchor sequence
LENGGTSLSEKTVLLLVTPFLAAAWSLHP

SEQ ID NO: 58 - protein, 18 aa, unidentified
  REGION 1..18: note = C-terminal targeting signal of K-Ras4B
SKDGKKKKKKSRTRCTVM